(19) United States
(12) Patent Application Publication
(10) Pub. No.: US 2026/0345287 A1
(43) Pub. Date: Nov. 5, 2026

(54) METHOD AND APPARATUS FOR PREVENTING MIGRATION OF TRANSPONDER TAG IN TISSUE

(71) Applicants: Hologic, Inc., Marlborough, MA (US); Somatex Medical Technologies GmbH, Berlin (DE)

(72) Inventors: Dirk HORNSCHEIDT, Berlin (DE); Nikolaos Argyrios GKANATSIOS, Newtown, CT (US); Sophie MELZER, Berlin (DE); Jonathan Cornelius BERNDT, Berlin (DE); Alonso ARCE GOMEZ, Alajuela (CR); Merlin Niels Alexander BERGMEISTER, Berlin (DE)

(73) Assignees: HOLOGIC, INC., Marlborough, MA (US); SOMATEX MEDICAL TECHNOLOGIES GMBH, Berlin (DE)

(21) Appl. No.: 19/123,634

(22) PCT Filed: Oct. 25, 2023
(86) PCT No.: PCT/US2023/077742

§ 371 Date: Apr. 23, 2025

Related U.S. Application Data
(60) Provisional application No. 63/380,825, filed on Oct. 25, 2022.

(51) Int. Cl. A61B 90/00; A61B 90/98

(52) U.S. Cl. CPC A61B 90/39; A61B 90/98; A61B 2090/3908; A61B 2090/3991

(57) ABSTRACT

A marking system for implantation into tissue comprises a marker body having an outer surface, the marker body including a transponder configured to respond to electromagnetic radiation from an external source, and a microchip operatively connected to the transponder and configured to activate in response to the electromagnetic radiation; and an anti-migration device configured to receive at least a portion of the marker body and to engage tissue in which the marker body is implanted to resist movement of the marker body after implantation. A deployment device, system, and method for implanting the marking system includes the use of a device having handle portion and a cannula within which the marking system is disposed.

### CROSS-REFERENCE TO RELATED APPLICATIONS

This application claims priority to U.S. Provisional Application No. 63/380,825, filed Oct. 25, 2022, the entire contents of which is incorporated by reference herein.

### TECHNICAL FIELD

The present invention relates generally to localization devices and more specifically to methods and apparatus used to prevent migration of implantable localization devices such as, for example, transponder tags in tissue.

### INTRODUCTION

Before a biopsy or surgical procedure to remove a lesion within a breast, such as a lumpectomy procedure, the location of the lesion must be identified. For example, mammography or ultrasound imaging may be used to identify and/or confirm the location of the lesion before a procedure. The resulting images may be used by a surgeon during a procedure to identify the location of the lesion and guide the surgeon, e.g., during dissection to access and/or remove the lesion. To facilitate localization, immediately before a procedure, a wire may be inserted into the breast, e.g., via a needle, such that a tip of the wire is positioned at the location of the lesion. Once the wire is positioned, it may be secured in place, e.g., using a bandage or tape applied to the patient's skin where the wire emerges from the breast. With the wire placed and secured in position, the patient may proceed to surgery, e.g., to have a biopsy or lumpectomy performed.

As an alternative to wires, radio frequency identification (RFID) tags, such as passive integrated transponder (PIT) tags, may be used to mark the position of a target tumor, lesion, body structure, or area within a patient's tissue, with the intent that the tag can be used to help a surgeon or clinician locate the structure, abnormality, or area at a later time for treatment. PIT tags provide increased precision and/or permit differentiation between multiple implanted devices. Each PIT tag generally includes a small ferrite-cored coil attached to a microchip enclosed in a glass covering or envelope. The microchip has a capacitor that causes the coil to resonate at a predetermined frequency when energized and circuitry to generate and transmit a coded identification number or message in response to a received interrogation signal which energizes the coil. Such PIT tags generally do not contain an internal energy source. Instead, energy needed to transmit the coded identification number is obtained through electromagnetic coupling, which causes a transfer of energy from a powered device to the PIT tag.

PIT tags may be injected up to a few centimeters (cm) below the outer surface of a patient's skin using a needle, cannula, or other suitable method of deployment to place the tag at or near the target with the intent that the tag can be used to help a surgeon or clinician locate the target at a later time for treatment. For example, such tags may be used to mark a lesion or tumor in the breast tissue for surgical removal. Neoadjuvant therapy, such as chemotherapy, radiation therapy, and hormone therapy, may be provided prior to surgical removal in order to reduce the size of the lesion or tumor. In such cases, the PIT tag may be used to mark the area for treatment and will remain in the tissue until a subsequent surgery to remove the lesion or tumor. In many applications, the embedded PIT tags are intended to remain in position for an extended period of time, such as between two procedures up to the life of the patient. In certain situations, it is beneficial to be able to quickly and accurately determine the location of the PIT tag in terms of the depth of the PIT tag relative to patient's skin. For example, knowing the exact position of a PIT tag is helpful to minimize the size of the incision necessary to remove the PIT tag from the patient.

However, the use of embedded PIT tags may present certain challenges to practitioners. If the PIT tag is stored in a deployment system (e.g., a cannula) prior to implantation, one must ensure that the PIT tag does not fall out of the deployment system during transport or storage. Challenges remain even after implantation. For example, the embedded PIT tags may migrate over time within the track of the needle or cannula used to insert the embedded PIT tags or in other directions. Such migration may occur immediately after deployment, after treatments such as neoadjuvant therapy, or over time after other procedures. Moreover, in some instances fault may occur in the deployment procedure, for example if the PIT tag fails to properly deploy from the needle. As a result, practitioners may find it difficult or impossible to locate the embedded PIT tags using imaging techniques such as ultrasound or magnetic resonance imaging (MRI) and/or to easily identify the embedded PIT tags using a one-image view. Additionally, if the PIT tag is used to mark the position of a structure, structural abnormality, or area within tissue and migrates away from the target location, it may be difficult to identify the structure, abnormality, or area within the tissue even when the PIT tag is located. An embedded PIT tag with low palpability (i.e., that is more difficult for the practitioner to locate by palpating the tag within the surgical cavity) may be more difficult or take more time for the practitioner to locate during a surgical procedure, for example when removing the PIT tag with the tumor or sentinel lymph node. Thus, there exists a need for an embedded PIT tag and deployment procedure that provides improved reliability, anchorage, visibility, and/or palpability for the practitioner.

### SUMMARY

In view of these and other circumstances, the present disclosure provides for methods, systems, and devices for preventing migration and increasing detectability of transponder tags.

In one aspect of the present disclosure, a marking system for implantation into tissue comprises a marker body having an outer surface, the marker body including a transponder configured to respond to electromagnetic radiation from an external source, and a microchip operatively connected to the transponder and configured to activate in response to the electromagnetic radiation; and an anti-migration device configured to receive at least a portion of the marker body and to engage tissue in which the marker body is implanted to resist movement of the marker body after implantation.

In another aspect of the present disclosure, a deployment system comprises a deployment device, including a handle portion including an actuator configured to cause a deployment operation, and a cannula attached to the handle portion at a first end of the cannula, wherein the cannula terminates in a cannula end at a second end of the cannula opposite the first end; and a marking system disposed within a lumen of the cannula, the marking system including a marker body having an outer surface, the marker body including a transponder configured to respond to electromagnetic radiation from an external source, and a microchip operatively connected to the transponder and configured to activate in response to the electromagnetic radiation, and an anti-migration device configured to resist movement of the marker body after implantation of the marking system in tissue.

In another aspect of the present disclosure, a method of marking a target location in a patient's body comprises positioning a distal end of a cannula adjacent to the target and underneath a tissue surface, wherein a tissue marker and an anti-migration device are disposed in a lumen of the cannula; deploying the tissue marker and the anti-migration device to the target location; and expanding a portion of the anti-migration device to engage tissue at the target location to anchor the tissue marker at the target location.

In another aspect of the present disclosure, a localization marker device for implantation into tissue comprises an implantable marker having a body with an outer surface, wherein the implantable marker is a transponder, a reflector, an active marker, a magnetic marker, a radioactive seed, a doppler marker, a passive marker, a wireless tumor location implant, or a combination thereof, and an anti-migration device configured to at least partially engage the outer surface of the body and to provide a movement resistance to the implantable marker after deployment.

Additional aspects of the present disclosure will be set forth in part in the description which follows, and in part will be obvious from the description or may be learned by practice of the present disclosure. The aspects of the present disclosure, and advantages which arise therefrom, may be realized and attained by means of the elements and combinations particularly pointed out in the appended claims and their equivalents.

It is to be understood that both the foregoing general description and the following detailed description are exemplary and explanatory only and are not restrictive of the present disclosure and claims.

### BRIEF DESCRIPTION OF THE DRAWINGS

These and other aspects of the present disclosure are described with respect to the attached drawings, in which:

FIG. 1 illustrates a side view of an exemplary PIT tag in accordance with various aspects of the present disclosure;

FIGS. 2A, 2B, and 2G respectively illustrate cross-sectional views of exemplary anti-migration devices in accordance with various aspects of the present disclosure;

FIGS. 2C-2F, and 2I respectively illustrate side views of exemplary anti-migration devices in accordance with various aspects of the present disclosure;

FIGS. 2H and 2J illustrates a perspective view of an exemplary anti-migration device in accordance with various aspects of the present disclosure;

FIGS. 3A and 3B respectively illustrate schematic views of an exemplary implantation device in accordance with various aspects of the present disclosure;

FIG. 4 illustrates a process flow of an exemplary loading method in accordance with various aspects of the present disclosure;

FIGS. 5A-5C respectively illustrate schematic views of the exemplary loading method of FIG. 4;

FIGS. 6A and 6B respectively illustrate schematic views of an exemplary implantation method in accordance with various aspects of the present disclosure;

FIGS. 7A and 7B respectively illustrate schematic views of another exemplary implantation device in accordance with various aspects of the present disclosure;

FIG. 8 illustrates a process flow of another exemplary loading method in accordance with various aspects of the present disclosure;

FIGS. 9A-9D respectively illustrate schematic views of the exemplary loading method of FIG. 8;

FIGS. 10A and 10B respectively illustrate schematic views of another exemplary implantation method in accordance with various aspects of the present disclosure;

FIGS. 11A and 11B respectively illustrate schematic views of another exemplary implantation device in accordance with various aspects of the present disclosure;

FIGS. 12A and 12B respectively illustrate schematic views of another exemplary implantation method in accordance with various aspects of the present disclosure;

FIGS. 13A and 13B respectively illustrate schematic views of yet another exemplary implantation method in accordance with various aspects of the present disclosure;

FIGS. 14A-14D respectively illustrate perspective views of exemplary sheathed tags in accordance with various aspects of the present disclosure;

FIGS. 14E-14L respectively illustrate side views of exemplary sheathed tags in accordance with various aspects of the present disclosure;

FIGS. 14M and 14N illustrate perspective views of an exemplary sheathed tag in accordance with various aspects of the present disclosure;

FIGS. 140 and 14P respectively illustrate a perspective view and an end view of another exemplary sheathed tag in accordance with various aspects of the present disclosure;

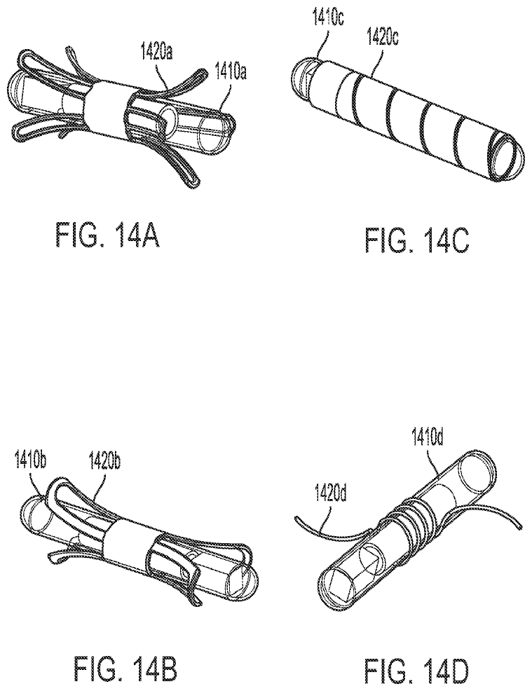

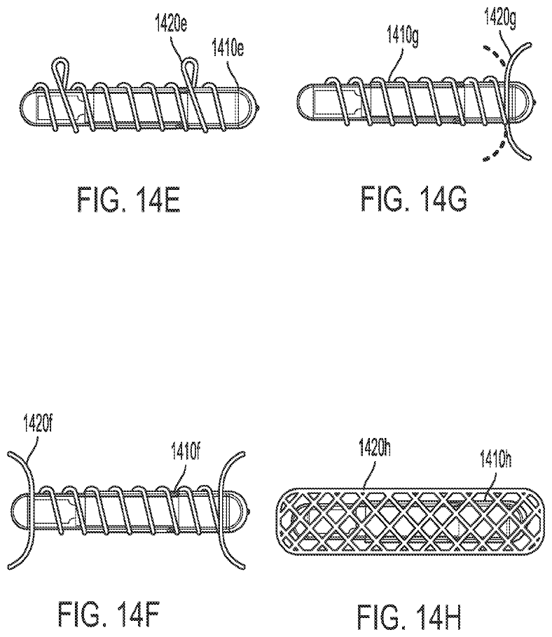

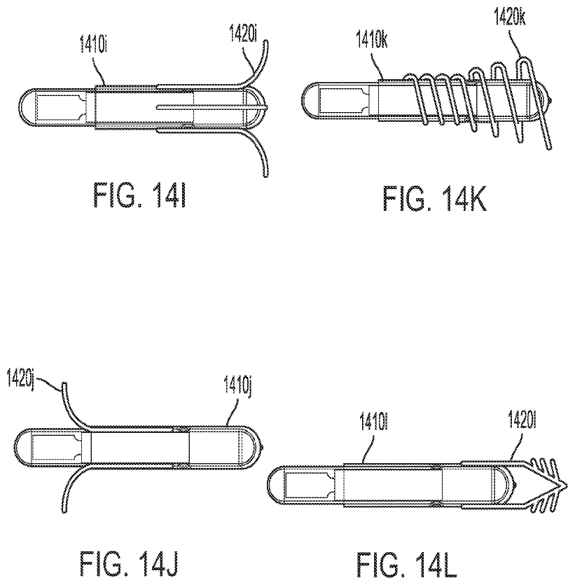

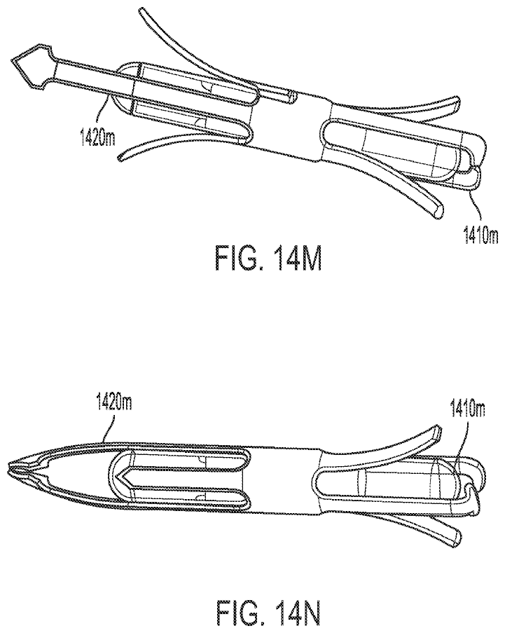

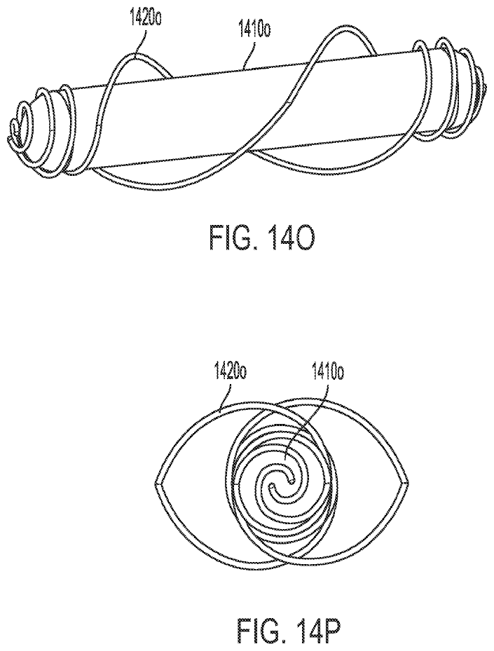

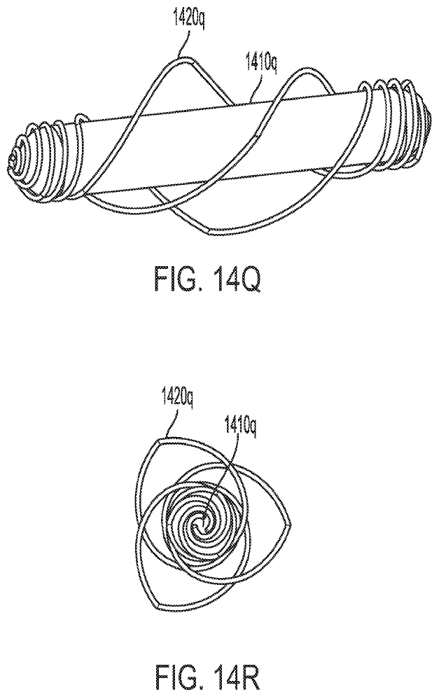

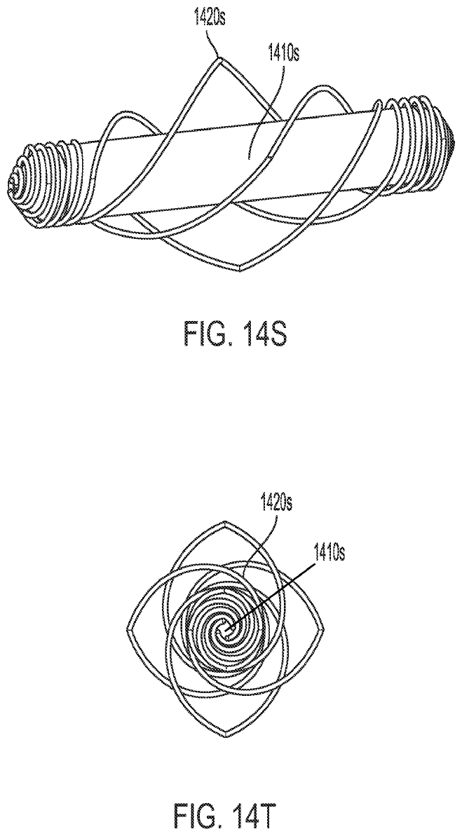

FIGS. 14Q and 14R respectively illustrate a perspective view and an end view of another exemplary sheathed tag in accordance with various aspects of the present disclosure; and

FIGS. 14S and 14T respectively illustrate a perspective view and an end view of yet another exemplary sheathed tag in accordance with various aspects of the present disclosure.

### DETAILED DESCRIPTION OF THE ILLUSTRATED EMBODIMENTS

PIT tags may be used to locate or identify the location of a tumor, lesion, body structure, or abnormality within a patient's tissue. For example, a PIT tag can be positioned within a patient's breast to mark a lesion for surgical removal and, if needed, for neoadjuvant therapy prior to surgical removal. The tag may be placed within breast tissue within about 6 cm of the breast surface to mark a lesion intended for surgical removal or other treatment. The tag can be surgically removed once treatment is complete or, in some cases, may permanently reside within the patient.

As noted above, PIT tags embedded in a patient may migrate over time, thus causing the embedded PIT tag to move from its expected location at the point of implantation. Migration may occur immediately after deployment, after treatments such as neoadjuvant therapy, or over time after other procedures. Although a position of the PIT tag may be detected using a handheld probe or reader (e.g., with sensors that are used to locate a signal emitted by the PIT tag) and may in some cases also be visually located (e.g., via ultrasound, x-ray, and/or MRI), migration of the PIT tag may make the process of locating the embedded PIT tag more difficult, for example, requiring that the clinician or surgeon take additional steps to locate the area that the PIT tag was originally intended to mark for treatment. Prior to the deployment process, the PIT tag is at risk of falling out of the deployment system during storage. The deployment process itself may not proceed properly if the PIT tag remains embedded in the deployment needle shaft. Additionally, deployment may be made more difficult (e.g., due to patient discomfort, increased risk of injury or scarring, and the like) if the cannula of the deployment system is not made as thin as possible. Such systems require the PIT tag to have small profiles (e.g., 2 mm) and, in some circumstances, the shape and small profile of the PIT tag may render the tag difficult to identify using a one-image view, for example in breast imaging.

Therefore, there exists a need for an implantable PIT tag and a deployment procedure that provide improved reliability, anchorage, visibility, and/or palpability for the practitioner. There also exists a need for a deployment system within which the PIT tag or other implantable object is securely held during storage, and which reduces the risk of negative effects on the patient. The present disclosure provides marking systems in which the PIT tag is combined with an anti-migration feature or component (e.g., a sheath or a wire as will be described in more detail below) to prevent migration of the PIT tag from a target location. The anti-migration feature or component may be a structure that changes an effective size and/or shape and/or outer contour of the PIT tag and may be formed, at least in part, from a material that is visible under inspection by one or more imaging modalities such as ultrasound. The anti-migration feature or component may have a low profile in the cannula and, due to its material properties, may expand upon deployment to prevent the assembly from migrating. As such, the present disclosure provides systems and methods which address the above-noted factors and provide for reliable deployment of implantable PIT tags, improved anchorage and anti-migration properties, enhanced visibility under various imaging techniques, and improved palpability for a practitioner.

The present disclosure provides several examples of use cases for the disclosed systems, methods, devices, and apparatus. Particular use cases are presented in the area of breast imaging and/or lumpectomies, but the present disclosure is not so limited. In general, the systems, methods, devices, and apparatus described herein may be applied in any situation where an object is implanted underneath a surface and where it would be beneficial to reduce or prevent the migration of the embedded/implanted object, to improve the reliability of the deployment procedure, and/or to improve the detectability of the object after deployment. For example, while the following detailed description is presented with regard to the implantation of a PIT tag, in practice the following description may be implemented as systems, methods, and apparatus for implanting an implantable object, for loading or otherwise preparing a deployment system of an implantable object, and deploying an implantable object from a deployment system, in which the implantable object is an object other than a PIT tag, such as a localization marker which may include but is not limited to transponders, reflectors, other active markers, magnetic markers, radioactive seeds, doppler markers, other passive markers, and/or other wireless (“non-wire”) tumor location implants, including combinations thereof. Each of these types of implantable objects may be implanted by inserting a cannula near a target location (i.e., a lesion or a tumor), deploying the implantable object from the cannula as described below, and removing the cannula; thus, these implantable objects may similarly be prone to migration after implantation as described above. Accordingly, references to a “PIT tag” below may generally be understood to also apply to any implantable object, and the present disclosure is generally applicable to a marking system for implantation into tissue in which the marking system includes a body to be implanted and an anti-migration device to prevent migration of the body.

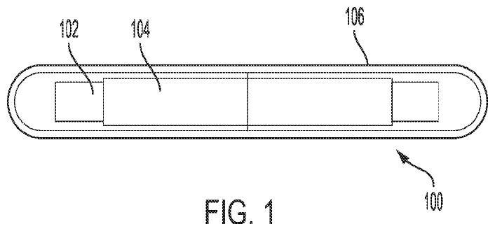

FIG. 1 illustrates an exemplary marker body, which is shown as a PIT tag 100, in accordance with the present disclosure, which may be one example of the body component of a marking system in accordance with the present disclosure. The PIT tag 100 includes a PIT 102 attached to a microchip 104 operatively connected to the PIT 102, both of which are encapsulated in an implantable shell 106 which defines the outer surface of the PIT tag 100. The PIT 102 is a passive device which has no battery or other power source; accordingly, the microchip 104 remains inactive until energized by electromagnetic radiation (e.g., radio or other low frequency energy) from an external source, such as a locator device. The microchip 104 in the PIT tag 100 may be prerecorded with a unique code, such as an alphabetic, numeric, or alphanumeric code, which may serve to identify a patient and/or a surgical procedure to be performed, for example. The PIT 102 may include an inductive coil which responds to an external signal at a resonant frequency of the inductive coil, thereby providing power needed to interrogate the microchip 104 to respond with its unique code back to the source of the external signal (e.g., the locator device), and thereby to positively identify the PIT tag 100 and provide its approximate location within the patient.

In order to permit the PIT tag 100 to be implantable within a patient, the implantable shell 106 may be made of a material that is sterilizable. In an example, the implantable shell 106 is capable of undergoing ethylene oxide (ETO) sterilization although other types of sterilization may be possible or suitable. The implantable shell 106 may also be of sufficient strength and/or durability to permit implantation for a desired period of time, such as for up to thirty days. In some implementations, the PIT tag 100 and implantable shell 106 may be permanently implantable. The implantable shell 106 may be made of a material that is resistant to cracks or splits. The material may be biocompatible. The implantable shell 106 may be made of a material that is transparent to radio or other low frequencies, at least in an area of the resonant frequency PIT 102 and/or the response frequency of the microchip 104. The implantable shell 106 may be made of glass or plastic, for example. In some examples, at least one of the PIT 102, the microchip 104, and the implantable shell 106 may be visible under ultrasound inspection. The implantable shell 106 must also be of a dimension (e.g., diameter, length, or both) to permit the PIT tag 100 to fit within the cannula or other device used to implant the PIT tag 100. In the particular example illustrated in FIG. 1, for example, the implantable shell 106 has a length of approximately 11 mm (0.4 in.) and a maximum diameter of approximately 2 mm (0.07 in.). As illustrated in FIG. 1, the implantable shell 106 has a two-part construction, although a unitary shell is also contemplated.

While FIG. 1 illustrates the implantable shell 106 as a generally smooth cylinder, one or more components having surface features may be utilized in order to increase resistance of the PIT tag 100 to movement; for example, by increasing the effective surface area of the implantable system and thus increasing frictional forces which would need to be overcome before any movement of the implantable system could occur. The surface feature(s) may be directly incorporated into a design of the implantable shell 106, for example by grooving, or the surface feature(s) may be incorporated as a separate element or sheath, referred to herein as an anti-migration device, which engages or receives the shell 106 either prior to or subsequent to implantation of the PIT tag 100, to result in a final implanted PIT assembly with anti-migration characteristics. An anti-migration device may comprise a structure that changes an effective size and/or shape and/or outer contour of the PIT tag (shell 106) to prevent movement or migration of the PIT tag from an implanted or deployed position. Thus, an anti-migration device may be configured with a portion having an inner surface that, at least to an extent, conforms to an outer surface of the PIT tag and a portion having an outer surface that, at least to an extent, expands or otherwise changes an effective outer contour, size, or shape of the outer surface of the PIT tag as the tag is deployed and positioned within the patient's tissue. The inner and outer surfaces described above may be on different portions of the anti-migration device or on the same portion of the anti-migration device. In some example embodiments, the anti-migration device may include structure(s) that act as barbs or anchors to engage the tissue, may include portions that expand to press against the tissue to resist movement, may include portions that provide increased friction against the tissue to resist movement, and the like. For example, the anti-migration device may include a nitinol wire with one or more barbs.

The surface feature(s) may also include elements which assist in the detection and location of the anti-migration device by touch (such as flanged portions, portions of an easily identifiable shape such as a ball or sphere, and the like) or under one or more imaging technologies (such as portions made of a material having high visibility under MRI, x-ray imaging, ultrasound, and the like). In some implementations, a single feature may be both an anti-migration and detection-and-location feature, both an anti-migration and imaging feature, or all three of an anti-migration, detection-and-location, and imaging feature. For example, the surface feature may include a ball which engages the tissue to prevent movement of the PIT tag 100 and simultaneously enhances the palpability of the marking system when implanted.

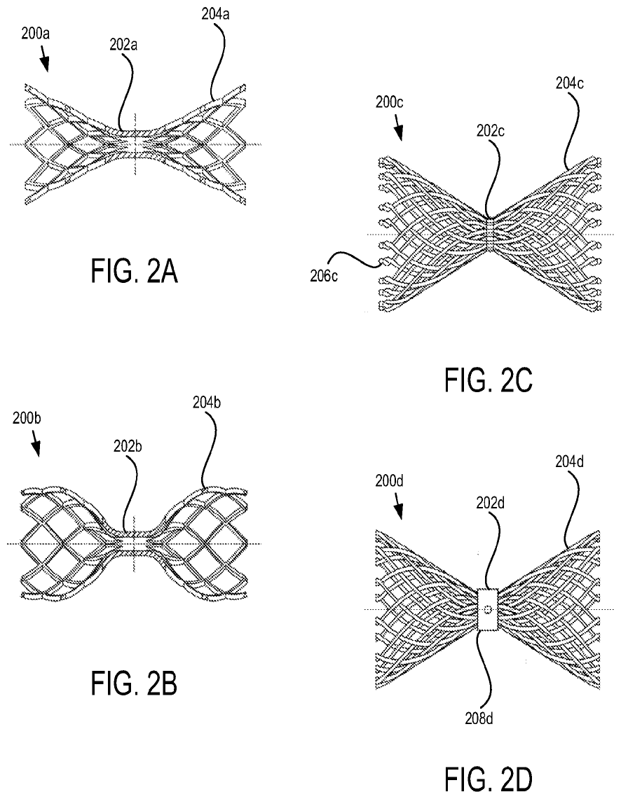

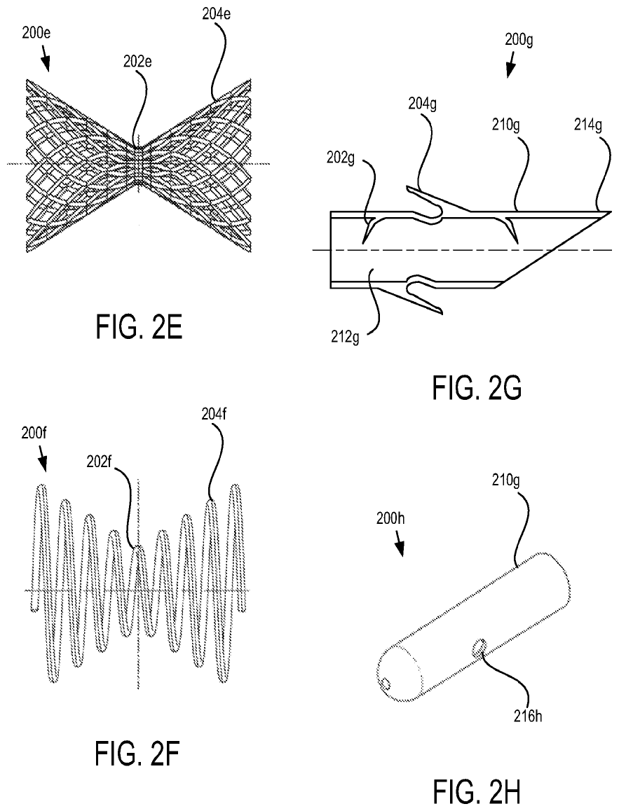

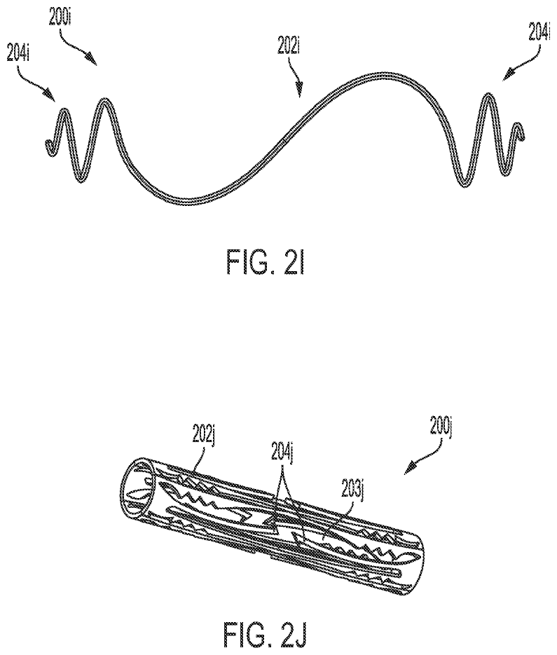

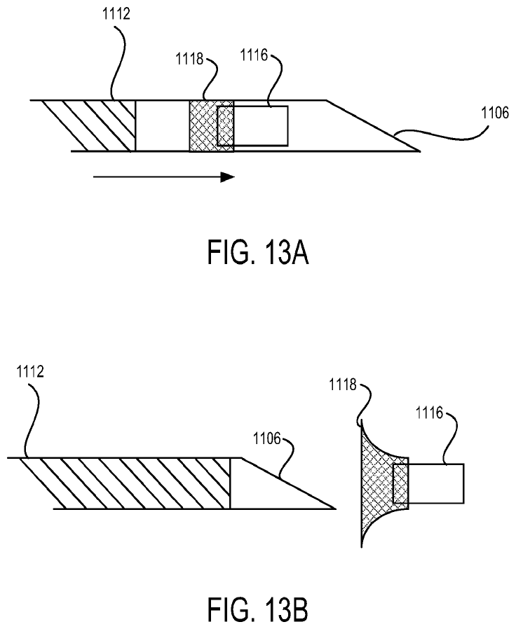

In addition to or as an alternative to the barb, protrusion, or anchor-type of surface features, the surface feature(s) may result in portions of the marking system, once implanted, having a comparatively large diameter (e.g., which provide an expansion force against tissue in a radial direction) and portions of the implanted system having a comparatively small diameter. In some example embodiments, the surface feature(s) may be implemented as an anti-migration device having a generally hourglass or bow-tie shape, with the larger diameter portions being located at the ends of the sheath in the axial (longitudinal) direction (and thus positioned toward the respective ends of the shell 106) and the small diameter portions being located at the center of the sheath in the axial direction. Several examples of anti-migration devices shaped to increase movement resistance of the marking system are described herein, including anti-migration devices having large diameter portions as shown in FIGS. 2A-2F, 13E, 13H, and 13K, anti-migration devices having radial protrusions as shown in FIGS. 2G, 13A-13B, 13D, 13G-13F, 13I-13J, and 13M, and anti-migration devices having sharpened points as shown in FIGS. 2G and 13L-13M.

As described in the examples herein, the shape of the shell 106 and/or anti-migration device may have a circular cross section which allows a comparison of diameters of cross sections taken along various axial locations of the device. It should be recognized that the present disclosure contemplates that PIT tag and/or the anti-migration device may have a different shape when viewed in cross section, including but not limited to an oval, a triangle, a square, a higher-order regular polygon, a concave polygon such as a star having any number of points, or combinations thereof. In such cases, the resulting cross sections would correspondingly be sized along the axial length of the anti-migration device.

An anti-migration device in accordance with the present disclosure can be formed of a non-resorbable material so that it may remain in tissue for a relatively long period of time without deterioration and without interacting with the adjacent tissue. In other implementations, the anti-migration device may be formed of a resorbable material, which may improve tissue ingrowth. The anti-migration device in accordance with the present disclosure may also be designed to improve detectability (e.g., improved ultrasound visibility, improved palpability, and the like). To aid in visibility in the case of sonography with, for example, medical ultrasound at frequencies ranging from 1 megahertz (MHz) to 40 MHz in a brightness-modulation mode, the anti-migration device may have a braided, woven, mesh, or webbed structure. The parameters of the web, such as the webbed body diameter (or width and thickness), web number, web density, and web material may be selected to match the acoustic energy of the incident ultrasound radiation. Ultrasound visibility may further be enhanced by roughening the webbed body, such as by sandblasting. Visibility may additionally or alternatively be enhanced by coating the webbed body. The coating may be a nanocoating, such as gold nanoparticles. To aid in visibility in the case of x-ray imaging methods in, for example, mammography, the anti-migration device may be formed of a material that provides good recognizability under x-ray examination. Thus, the webbed body may be formed of a metal or metal alloy, for example by metal wires or by metal particles embedded in plastic. The metal may be one exhibiting magnetic properties, which may improve recognizability under MRI examination. The metal or metal alloy may include titanium, gold, and/or iron. In one particular example, the anti-migration device is formed from a titanium alloy such as nitinol.

The anti-migration device may alternatively be formed of a material that permits manufacture using an injection molding method. Thus, in addition to a metal, the anti-migration device may also be formed of a plastic or rubber material, including but not limited to silicone, ethylene propylene diene monomer (EPDM), thermoplastic elastomer (TPE), polyether ether ketone (PEEK), or polypropylene (PP). In some implementations, certain wires of the webbed body may be formed of a metal while other wires of the webbed body are formed of a plastic or rubber. Such materials may also provide shape-memory benefits, thus allowing the anti-migration device to exist in a compressed state (e.g., in a cannula of the implantation device) for an extended period of time and retain the ability to regain its expanded state upon implantation. The anti-migration device may therefore be designed to achieve elastic compression under a radial force of at least one newton (N); thereafter, upon implantation in its elastically compressed state, the anti-migration device may independently transition to its expanded state and retain this state so long as the surrounding tissue exerts a radial force of 1 N or less (e.g., 0.1 N) on the anti-migration device. The anti-migration device may have a first size and a first shape in the compressed state and a second size and a second shape in the expanded state, wherein the first size may be different than the second size and/or the first shape may be different than the second shape. As will be described in more detail below, however, in some implementations the anti-migration device may include geometric features which cut into the tissue while the implant is pushed out of the cannula, which may further reduce (or even eliminate) the required radial force. In its compressed state, an external diameter of the anti-migration device may be sufficiently small to permit the anti-migration device to reside in the same cannula as the PIT tag. In its expanded state, an internal diameter of the anti-migration device may be sufficiently large to permit the PIT tag to be retained by the anti-migration device.

The anti-migration device may be made of a material that is sterilizable (e.g., capable of undergoing ETO sterilization) in order to permit the anti-migration device to be implantable. The anti-migration device should also be of sufficient strength and/or durability to permit implantation for a desired period of time. In some implementations, the PIT tag 100 and implantable shell 106 may be permanently implantable. In implementation where the anti-migration device is sufficiently large that, in the deployed state, it covers RF-responsive portions of the PIT tag 100, the anti-migration device may be made of a material that is transparent to radio or other low frequencies.

FIGS. 2A-2J respectively illustrate exemplary anti-migration devices 200a-200j in accordance with the present disclosure (collectively referred to as “anti-migration device 200” where it is not necessary to distinguish therebetween). FIGS. 2A, 2B, and 2G show cross sectional views, whereas FIGS. 2C-2F, and 2I show side views and FIGS. 2H and 2J each show a perspective view.

As shown in FIG. 2A, an anti-migration device 200a includes a small diameter portion 202a located at approximately the center thereof in an axial (length) direction, and two large diameter portions 204a located at each end thereof in the axial direction. The small diameter portion 202a may be dimensioned so as to frictionally engage with a marking element received in the anti-migration device 200a (e.g., a PIT tag as will be described in more detail below). The large diameter portion 204a may be dimensioned so as to provide a frictional anti-migration force to resist movement in the medium in which the anti-migration device 200a is implanted (e.g., tissue as will be described in more detail below) and operates as an anti-migration feature. As embodied in FIG. 2A, each end of the anti-migration device 200a may terminate in a plurality of points, shown in FIG. 2A as eight points. In the illustrated example, the anti-migration device 200a is generally rotationally symmetric, has a generally circular footprint when viewed head-on (i.e., from a plane perpendicular to the axial direction), and has a generally webbed construction.

FIG. 2B illustrates an anti-migration device 200b which is similar to the anti-migration device 200a at least in that it includes a small diameter portion 202b located at approximately the center thereof in an axial direction and two large diameter portions 204b located at each end thereof in the axial direction. The small diameter portion 202b is dimensioned so as to frictionally engage with a marking element received within the anti-migration device 200b, and the large diameter portions 204b are dimensioned so as to provide a frictional anti-migration force to resist movement in the medium in which the anti-migration device 200b is implanted.

FIG. 2C illustrates an anti-migration device 200c that also includes a small diameter portion 202c located at approximately the center thereof in an axial direction with two large diameter portions 204c located at opposite axial ends of the component. The small diameter portion 202c is also dimensioned so as to frictionally engage with a marking element received within the anti-migration device 200c, and the large diameter portions 204c are dimensioned so as to provide a frictional anti-migration force to resist movement in the medium in which the anti-migration device 200c is implanted. In the event that frictional force is not sufficient to fix the marking element within the small diameter portion 202c, an adhesive or other fastening method may be used. As also shown in the embodiments of FIGS. 2A and 2B, the end portions of the anti-migration component 200c may include a number of points (as illustrated, twelve). The number of end points provided on each axial end (large diameter portions 204c) may vary depending, for example, on the size of the end, the number of wires or webs forming the end, the type of material used, etc. A larger number of end points may provide increased anti-migration characteristics compared to an anti-migration device having a smaller number of end points, for example by increasing the number of contact points between the sheath and the tissue and/or by increasing the expansion force of the sheath. A larger number of wires in the braid may also increase visibility under ultrasound imaging. Moreover, in one example embodiment, each large diameter end portion has an end feature 206c, which in the illustrated example is ball-shaped. The end feature 206c may be formed by welding or twisting adjacent wires of the anti-migration device 200c. The end features 206c may be of various shapes and/or sizes and may provide improved palpability for a practitioner.

The anti-migration device 200d shown in FIG. 2D includes a central constrictor 208d, which may assist with maintaining the diameter of the small-diameter portion 202d. The central constrictor 208d may further assist in maintaining the structural integrity of the anti-migration device 200d, such as by holding individual wires together in a clamping fashion. The central constrictor 208d may be formed of the same material as the anti-migration device 200d or of a different material, and may include air/gas inclusions to increase recognizability under ultrasound imaging.

The anti-migration devices 200a-200b are formed by laser-cutting tubes and the anti-migration devices 200c-200d are formed generally by braiding wires. The anti-migration device 200e illustrated in FIG. 2E, on the other hand, is formed by welding wires together at the crossing points thereof. This may enhance the rigidity of the anti-migration device 200e in its expanded state, such that a relatively constant transition between the small-diameter portion 202e and the large-diameter portions 204e is achieved.

As an alternative to the webbed/braided/woven body designs of the anti-migration devices 200a-200e, the anti-migration devices 200f and 200i illustrated in FIGS. 2F and 2I have spiral designs. With reference to the embodiment of FIG. 2F, a spiral body of the anti-migration device 200f includes a small diameter portion 202f located at approximately the center thereof in an axial direction and two large diameter portions 204f located at each axial end of the anti-migration device 200f. In this manner, each of the anti-migration devices 200a-f illustrated in FIGS. 2A-2F have a form factor that is generally hourglass shaped in a side view (or has two funnel-shaped portions with each of the funnel-shaped portions tapering from a large axial cross section toward a small axial cross section at a central portion of the anti-migration device, which may also be considered an hourglass shape as used in the present disclosure). An anti-migration device 200 in accordance with the present disclosure may be formed with a cone angle (i.e., an angle between the axis and the cone surface) between 25° and 50°, in particular between 30° and 45°. Where the webbed shape is formed by braiding wires, the wire diameter is less than 0.5 mm (0.02 in.), for example, it may be less than or equal to 0.1 mm (0.004 in.), and in one particular example is between 0.04 mm (0.016 in.) and 0.10 mm (0.0039 in.). A wire diameter within these ranges may enhance the compressibility of the anti-migration device 200, while enhancing the expansion force of the anti-migration device 200 permitting it to expand against tissue pressure that may be prevalent in hard tissue such as tumor tissue.

With reference to the embodiment of FIG. 2I, in contrast to the anti-migration device 200f, the anti-migration device 200i includes a spiral body including a large diameter portion 202i located along a middle portion of the anti-migration device 200i in an axial direction and two small diameter portions 204i located at each axial end of the anti-migration device 200i. In such embodiments, the spiral body can be formed from one or more wires that are made from a shape memory material, such as, for example, nitinol (one wire being shown in the embodiment of FIG. 2I). In this manner, when the anti-migration device 200i is pushed out of a delivery device (e.g., cannula), as described further below, the middle portion of the anti-migration device self-expands to form the large diameter portion 202i (i.e., to prevent migration of the PIT Tag held within the anti-migration device 200i), while each of the small diameter portions 204i grip the rounded ends of the PIT tag (see, e.g., FIGS. 140-14T).

As an alternative to the flared body design of the anti-migration devices 200a-200f, the anti-migration device 200g illustrated in FIG. 2G has a more cylindrical design with branched portions or protrusions, the anti-migration device 200h illustrated in FIG. 2H has a cap-like design, and the anti-migration device 200j illustrated in FIG. 2J has a cylindrical design with cut-outs formed by micro injection molding and/or laser cutting. The anti-migration devices 200g and 200h are, for example, configured to provide a slimmer profile than the embodiments of FIGS. 2A-2F and thus are not intended to expand or otherwise change shape upon deployment. The use of designs that do not change shape permits the use of a wider range of materials for the anti-migration device 200g, 200h as compared to the anti-migration devices 200a-200f. The anti-migration device 200j takes advantage of processes, such as micro-injection molding and laser cutting, to also utilize additional materials, such as, for example, plastic materials, as described further below.

As shown in FIG. 2G, an anti-migration device 200g includes a hollow body 210g which defines an interior 212g of the anti-migration device 200g. The hollow body 210g is configured with external protrusions 204g extending from an outer surface of the hollow body 210g and internal protrusions 202g extending from an inner surface of the hollow body 210g into the interior 212g. The interior 212g is sized to receive a marking element and the internal protrusions 202g may frictionally engage the marking element when the marking element is positioned in the interior 212g. The internal protrusions 202g may extend radially from the inner surface toward a center axis of the anti-migration device 200g and may form any pattern and include any number of protrusions. The internal protrusions 202g may extend perpendicularly and/or at an angle from the inner surface of the hollow body 210g. Similarly, external protrusions 204g may extend radially outward from the outer surface and may form any pattern and include any number of protrusions. The external protrusions 204g may be positioned to engage tissue surrounding the hollow body 210g after implantation and may include pointed ends to facilitate engagement so as to resist movement of the anti-migration device 200g once implanted. The external protrusions 204g may extend perpendicularly and/or at an angle from the outer surface of the hollow body 210g. The external protrusions 204g and/or the internal protrusions 202g may be produced by cutting (e.g., laser cutting) a U, V, or similar shape into the outer surface or inner surface of the hollow body 210g. While two external protrusions 204g and two internal protrusions 202g are illustrated in FIG. 2G, in practical implementations the anti-migration component 200g may include additional or fewer of external protrusions 204g and/or internal protrusions 202g. The external protrusions 204g and/or internal protrusions 202g may be regularly or irregularly spaced in a circumferential direction. As shown in FIG. 2G, one end of the anti-migration device 200g terminates in a sharpened point 214g, which may have the function of a cannula to improve penetrability into hard tissues, such as tumors, cartilage, or bone. FIG. 2G illustrates the anti-migration device 200g in its expanded state, with the external protrusions 204g in a radially extending position (i.e., after implantation). Prior to implantation, the external protrusions 204g may be in an axially extending position, such that the external protrusions 204g move from the axially extending position to the radially extending position during implantation.

The cap-like anti-migration device 200h may partially or fully receive a marking element, such as a PIT tag, in an interior of a hollow body 210g forming the anti-migration device 200h. The marking element may be secured to the hollow body 210g in a heated state, such that the hollow body 210g holds the marking element in an airtight manner after the anti-migration device 200h cools. Such an attachment procedure permits a secure attachment without glue or other adhesives. The anti-migration device 200h further includes a plurality of side holes 216h which may assist with mounting, although only one side hole 216h is visible in the perspective view of FIG. 2H.

With reference now to FIG. 2J, a plastic structure 202j of an anti-migration device 200j can be created, using, for example, a micro injection molding process, that includes a plurality of cut-outs 203j forming a plurality of barbs 204j. Although it is difficult to store most plastic materials under tension (e.g., like Nitinol), since over time the plastic material will take on the shape of the structure in which it is stored (i.e., thereby making self-expansion not possible), the plastic structure 202j exemplifies a design which overcomes this problem.

For example, in such embodiments, the barbs 204j are configured to be under tension when the anti-migration device 200j is elongated (e.g., the anti-migration device 200j can be pre-tensioned via a stop on a tongue of the cannula as the anti-migration device 200j is loaded into the cannula), such that the barbs 204j expand radially outward when the anti-migration device 200j leaves the cannula. Thus, the plastic structure 202j is pre-tensioned in the cannula immediately during implantation, and the preload is fixed by a locking mechanism, exemplified by the barbs 204j. The cylindrical plastic structure 202j is compressed, for example, until the hooks 204j click into place, and the struts inside the cannula deform elastically along the inner wall of the cannula. In this manner, when the pre-stressed anti-migration device 200j leaves the cannula, the struts straighten up and increase the volume of the anti-migration device 200j so that it no longer fits through the puncture channel (i.e., created by the cannula). Those of ordinary skill in the art will understand that the cut-outs 203j and the formed plastic barbs 204j illustrated in the embodiment of FIG. 2J are exemplary only that the contemplated anti-migration devices 200j can include cut-outs 203j of various shapes and sizes, which form barbs 204j having various shapes and sizes, and which create various different patterned structures 202j.

In the expanded state, a maximum external diameter of an anti-migration device 200a-200f, 200i, and 200j, in accordance with the present disclosure (e.g., an external diameter of large-diameter portions 204a-204f) may be less than 10 mm (0.4 in.), in some cases less than 8 mm (0.3 in.), and in example embodiments is between 3.0 mm (0.12 in.) and 5.0 mm (0.20 in.). Such a diameter retains good visibility under several imaging methods while retaining a sufficiently small spatial requirement for a foreign body in tissue. Also, in the expanded state, a minimum internal diameter of the anti-migration device 200a-200f, 200i, and 200j in accordance with the present disclosure (e.g., an internal diameter of small-diameter portions 202a-202f, 200i, and 200j) may be approximately equal to an external diameter of the marking element such as PIT tag 100 to permit the anti-migration device 200 to retain the PIT tag 100 therein. In the compressed state, on the other hand, the outer diameter of the anti-migration device 200a-200f, 200i, and 200j should be only slightly larger than the external diameter of the marking element such as PIT tag 100. The anti-migration device 200a-200f, 200i, and 200j may have an external diameter of less than or equal to 3 mm (0.1 in.), and in some example embodiments less than or equal to approximately 2 mm (0.08 in.). The anti-migration device 200 may also have a wall or braid thickness of less than or equal to 0.3 mm (0.01 in.); for example, 0.1 mm (0.004 in.). In one particular example, the PIT tag 100 may have an outer diameter of 1.41 mm (0.0555 in.), and the anti-migration device 200 has a wall thickness of 0.1 mm (0.0004 in.) and a minimum diameter in the compressed state of 1.61 mm (0.634 in.). In embodiments in which the anti-migration device does not change shape or size, such as anti-migration components 200g, 200h, the outer diameter of the anti-migration device should be only slightly larger than the external diameter of the marking element such as PIT tag 100. The anti-migration device 200g, 200h may have an external diameter of less than or equal to 3 mm (0.1 in.), and in some example embodiments less than or equal to approximately 2 mm (0.08 in.). A minimum internal diameter of the anti-migration device 200g, 200h in accordance with the present disclosure (e.g., an internal diameter of hollow body 210g, 210h) may be approximately equal to an external diameter of the marking element, such as PIT tag 100, to permit the anti-migration device 200 to retain the PIT tag 100 therein.

Individual elements of the anti-migration devices 200 may be selected and combined. For example, an anti-migration device 200 in accordance with the present disclosure may include both end features (similar to end features 206c illustrated in FIG. 2C) and a central constrictor (similar to central constrictor 208d illustrated in FIG. D). In another example, an anti-migration device 200 in accordance with the present disclosure may have an overall form factor similar to the anti-migration device 200a or 200b but with the larger number of end points possessed by the anti-migration device 200c or 200d. In yet another example, an anti-migration device 200 in accordance with the present disclosure may be combined with the cap-like anti-migration device 200h illustrated in FIG. 2H. For example, if an anti-migration device 200 is formed of wires, the wires can be melted into the surface of the anti-migration device 200h to provide a more secure attachment.

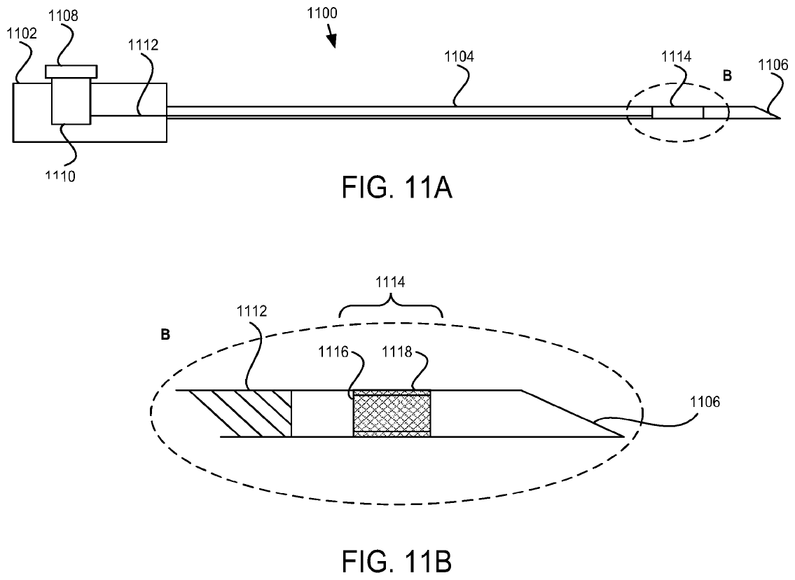

Moreover, while the anti-migration devices 200a-j illustrated in FIGS. 2A-2J are generally circular when viewed in an axial direction (i.e., have a circular axial cross section), an anti-migration device 200 in accordance with the present disclosure may have a different axial cross-sectional shape, including but not limited to an oval, a triangle, a square, a higher-order regular polygon, a concave polygon such as a star having any number of points, or combinations thereof. In some implementations, the axial cross-sectional shape may be irregular (e.g., not symmetric). The individual faces of the axial cross-sectional shape may be generally linear, curved, curvilinear, or combinations thereof. Other possible shapes for the anti-migration device 200 are illustrated in FIGS. 11A-D (in a deployed state) and will be described in more detail below.

While each of the anti-migration devices 200a-200j are illustrated as being of a generally unitary construction, an anti-migration device 200 in accordance with the present disclosure may be formed of two or more separate parts. For example, an anti-migration device 200 in accordance with the present disclosure may be formed of two parts, each of which has a small diameter portion 202 at one end and a large diameter portion 204 at the other end. The two parts may be arranged front-to-back, such that the small diameter portions 202 face toward one another and the large diameter portions 204 face away from one another. In such an arrangement, the center of the combined parts will be generally smaller in diameter than the ends of the combined parts.

In accordance with the present disclosure, a marking element such as a PIT tag (e.g., the PIT tag 100 illustrated in FIG. 1) and an anti-migration device (e.g., any of the anti-migration devices 200a-200j illustrated in FIGS. 2A-2J) may be implanted at substantially the same time. Generally, a method of implanting a PIT tag (or other implantable marker object) in accordance with the present disclosure may include providing a deployment system (also referred to as an implantation system); inserting a cannula end of the deployment system to a deployment location underneath a tissue surface; actuating a drive element of the deployment system to impart a force to the implantable object and/or the anti-migration device toward the cannula end; and thus implanting the implantable object at the deployment location such that at least a part of the implantable object is contained within the anti-migration device.

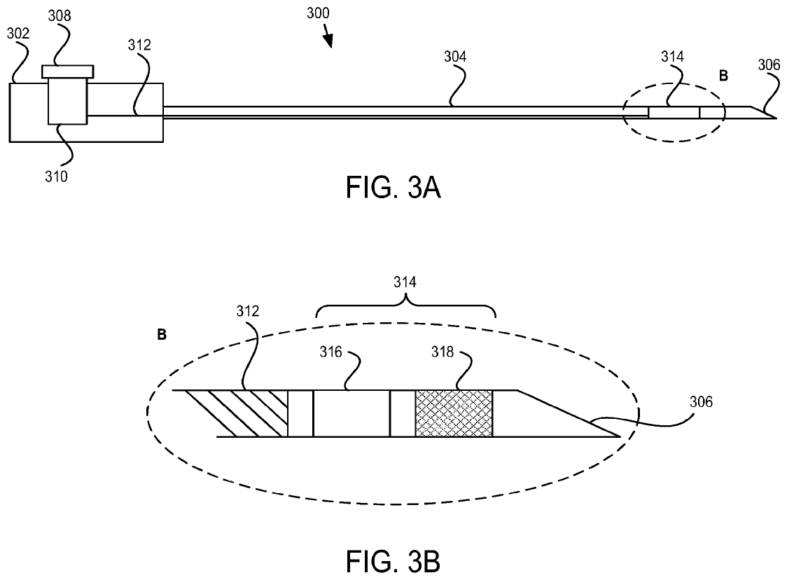

FIGS. 3A-3B illustrate a first exemplary implantation system 300 in accordance with the present disclosure, in a ready-to-deploy state. The implantation system 300 comprises a deployment device including a handle portion 302 and a cannula 304 including a lumen within which a deployment package 314 is disposed.

The handle portion 302 comprises a deployment button 308 or other actuator which operates a plunger 310, which in turn operates a driving element 312. The handle portion 302 and one or more components thereof may be produced from a suitable plastic. The deployment button 308 is connected to a housing of the handle portion 302 and is actuatable by a user, such as a surgeon. The deployment button 308 may physically be depressed or moved or may be a soft button configured to generate an electric or electronic signal upon actuation. The cannula 304 extends a distance away from the handle portion 302 and terminates in a cannula tip 306. The cannula 304 may be formed from a suitable metal. The cannula 304 has a length which may range from 25 mm (1 in.) to 200 mm (8 in.), and in some example embodiments is between 50 (2 in.) mm and 150 mm (6 in.). The length of the cannula 304 affects the range of the implantation system 300 in respect of the reachability of tissue sites in the body of a subject. A longer-length cannula 304 may be used when adjustment aids are used, for example for stereotaxis. The cannula 304 terminates in a cannula tip 306. The inner diameter of the cannula 304 may closely match the outer diameter of the deployment package 314 and may be approximately 0.15 mm (0.006 in.). For purposes of explanation, the direction of the cannula tip 306 is referred to herein as the “distal” direction and the direction of the handle portion 302 is referred to herein as the “proximal” direction.

The driving element 312 extends through the cannula 304. When the deployment button 308 is actuated, the plunger 310 operates to cause the driving element 312 to move in an axial direction within the cannula 304. The driving element 312 may be formed of a wire or a sufficiently stable plastic fiber. Consequently, the driving element 312 may be moved along a straight, guided sliding path between a pre-loaded position and a deployment position. In the deployment position, force from the plunger 310 is transferred to the deployment package 314 which has been pre-loaded at a location near to the cannula tip 306. Thus, when the deployment button 308 is actuated, the deployment package 314 may be driven out of the cannula 304 to the tissue site at the location of the cannula tip 306 by the axial movement of the driving element 312. This may be achieved by virtue of the driving element 312 being aligned coaxially with respect to the cannula 304 and hence pushing the pre-loaded deployment package 314 out of the cannula 304 past the cannula tip 306. One example of the deployment package 314 is shown in more detail in inset B, corresponding to FIG. 3B.

In FIG. 3B, the driving element 312 is in the pre-loaded position. The deployment package 314 includes a PIT tag 316 distal to the driving element 312 and proximal to an anti-migration device 318 and the anti-migration device 318 distal to the driving element 312 and PIT tag 316 and proximal to the cannula tip 306. The PIT tag 316 may be, for example, the PIT tag 100 illustrated in FIG. 1. As an alternative to the configuration shown in FIG. 3B, the deployment package 314 of FIG. 3A may be formed of a PIT tag 316 that is already disposed in anti-migration device 318, as described in more detail below with regard to FIGS. 11A-11B. By comparison, however, the configuration shown in FIG. 3B may permit the use of a cannula having a smaller diameter. The anti-migration device 318 may be, for example, any of the anti-migration devices 200a-200h illustrated in FIGS. 2A-2H or may be a modification or alternative thereto as described above. The anti-migration device 318 is in a compressed state. On account of pre-stress, the anti-migration device 318 (and thus the PIT tag 316) maintains its position within the cannula 304.

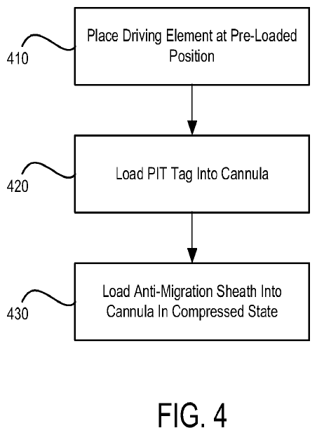

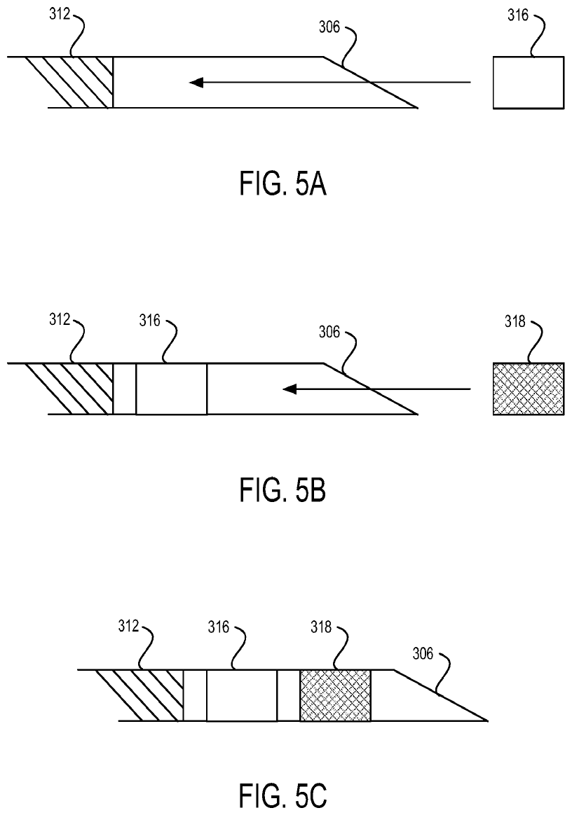

FIG. 4 illustrates an exemplary process flow for pre-loading the deployment package 314 in the deployment system 300. For purposes of explanation, the process flow will be described in the context of the deployment system 300 illustrated in FIGS. 3A-B. In this illustration, various processes within the process flow are illustrated in FIGS. 5A-5C. At operation 410, the process flow includes placing the driving element 312 at the pre-loaded position. Operation 410 may include actuating a button, such as the deployment button 308 or a reset/return button (not illustrated). In the event that the driving element 312 is already at the pre-loaded position, operation 410 may be omitted. Next, at operation 420, the PIT tag 316 is loaded into the cannula 304. The PIT tag 316 may be loaded via the cannula tip 306 or, if the proximal end of the cannula 304 is accessible, via the proximal end (e.g., before a portion of the handle portion 302 is attached). An example of operation 420 is illustrated in FIG. 5A, in which the PIT tag 316 is loaded via the cannula tip 306. The PIT tag 316 may be loaded such that a certain distance exists between the PIT tag 316 and the driving element 312. After operation 420, at operation 430 the anti-migration device 318 is loaded into the cannula 304, for example via the cannula tip 306. An example of operation 430 is illustrated in FIG. 5C. The anti-migration device 318 may be loaded such that a certain distance exists between the anti-migration device 318 and the PIT tag 316. After operation 430, the deployment system 300 is in the deployment-ready state as illustrated in FIG. 5C. It can be seen that the deployment-ready state illustrated in FIG. 5C corresponds to the inset of FIG. 3B.

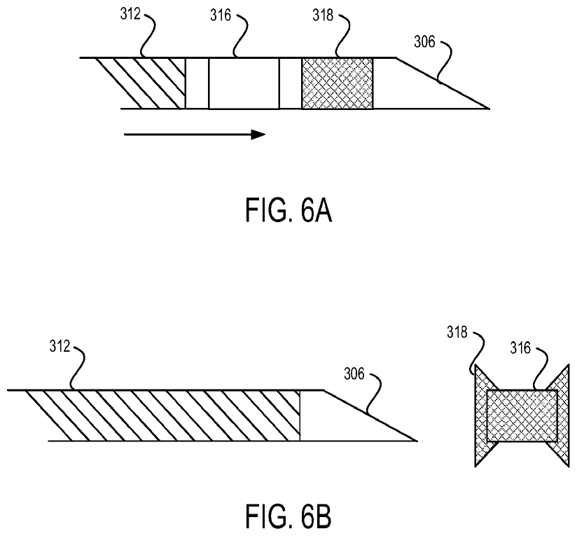

The deployment system 300 may exist in the deployment-ready state for an extended period of time (e.g., years) until the deployment package 314 is to be used. At such time, the deployment system 300 may be operated to simultaneously (or nearly simultaneously) deploy the entire deployment package 314. FIGS. 6A-6B illustrate the deployment procedure of the deployment system 300. Initially, as shown in FIG. 6A, the deployment system 300 is provided in the deployment-ready state. This state also corresponds to the state shown in FIGS. 3B and 5C. Subsequently, the operator (e.g., a surgeon) actuates the deployment button 308 causing the driving element 312 to move from the pre-loaded position to a deployment position. In this position, as shown in FIG. 6B, the driving element 312 has been extended to a point near the cannula tip 306. The contact force of the driving element 312 on the PIT tag 316 and thus on the anti-migration device 318 causes the deployment package 314 to exit the cannula 304 as a sheathed PIT tag, in which the PIT tag 316 resides within the anti-migration device 318 and in which the anti-migration device 318 has returned to its flared shape. For illustration purposes, the deployed anti-migration device 318 is illustrated as having a longer length than the PIT tag 316, but in practical implementations the anti-migration device 318 may preferably be shorter or of equal length, such that it does not extend beyond the PIT tag 316 in either direction.

While FIGS. 6A-6B illustrate a deployment procedure in which the cannula tip 306 remains generally stationary relative to the deployment area while the driving element 312 and deployment package 314 move into the deployment area, in some implementations the converse may be true. In such implementations, the cannula 304 with the deployment package 314 may be advanced to the deployment site and, subsequently, the cannula 304 may be withdrawn (e.g., over a guidewire or the driving element 312). When the cannula 304 becomes sufficiently retracted that the cannula tip 306 clears the deployment package 314, the deployment package 314 will have been deployed into the deployment area. The cannula 304 may subsequently be fully withdrawn. The retraction of the cannula tip 306 may be automatically initiated in response to the actuation of the deployment button 308, or the operator may manually retract the cannula tip 306. Such a procedure may be beneficial in cases where hard tissue (e.g., a tumor) is located at the deployment area, as such a procedure may reduce lateral migration that would otherwise be caused by the force of the deployment package 314 against the hard tissue. In such a case, the cannula tip 306 itself may initially be used to puncture the hard tissue and thereby create a secure deposition location for the deployment package 314.

Generally, the PIT tag 316 may enter the anti-migration device 318 in one of two ways, depending on the relative physical parameters (e.g., relative diameters) of the cannula 304, the PIT tag 316, and the anti-migration device 318; and in some cases, on the relative stiffness of the tissue into which the deployment package 314 is deployed. In the first instance, the force of the driving element 312 pushes the PIT tag 316 into the compressed anti-migration device 318 within the cannula 304, such that the anti-migration device 318 securely holds the PIT tag 316. The force of the driving element 312 may then subsequently push both the PIT tag 316 and the anti-migration device 318 into the tissue simultaneously. In the second instance, the force of the driving element 312 causes the PIT tag 316 to itself exert a force on the anti-migration device 318, causing the anti-migration device 318 to enter the tissue and expand. The driving element 312 continues to exert a force on the PIT tag 316 such that it enters into and becomes securely held by the anti-migration device 318 within the tissue. In such an implementation, an inner diameter of a part of the anti-migration device 318 (e.g., an inner diameter of the small-diameter portions 202c, 202e, or 202f shown in FIGS. 2C, 2E, and 2F) may be smaller than the outer diameter of the PIT tag 316. The motion of the PIT tag 316 causes the small-diameter portion of the anti-migration device 318 to expand, and the spring force of the anti-migration device 318 holds the PIT tag 316 firmly in place.

In any of the above procedures, the deployment procedure may be guided through the use of an imaging technology. For example, an operator may monitor the implantation area to ensure that the PIT tag 316 and the anti-migration device 318 are properly positioned with respect to one another and with respect to the implantation area itself. Thus, the operator may ensure that the combined deployment package 314, upon deployment, is appropriately situated relative to the lesion, tumor, or other object whose location the PIT tag 316 is intended to mark.

Also, in any of the above pre-loading or deployment procedures, the deployment package 314 or the PIT tag 316 and anti-migration device 318 may be packaged with a lubricant that eases the movement of the package 314 or the PIT tag 316 and anti-migration device 318 relative to the cannula 304. Such a lubricant may include a biocompatible silicon-based lubricant.

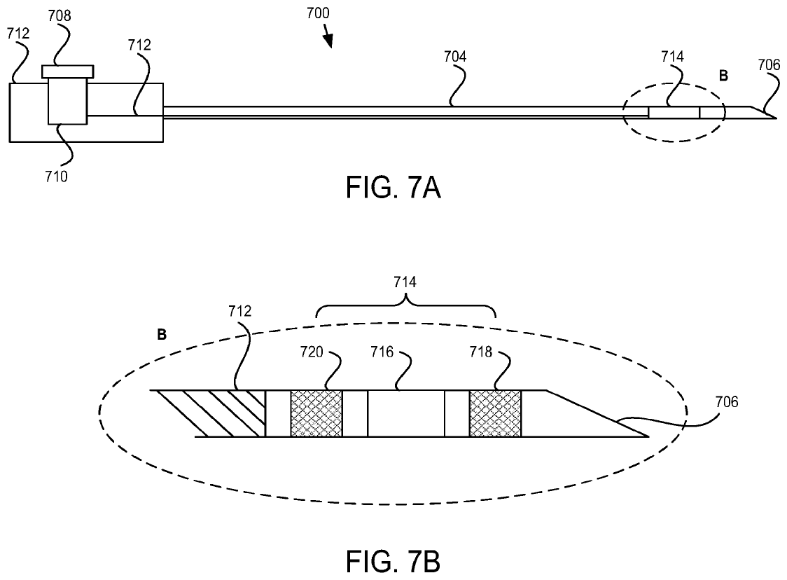

FIGS. 7A-7B illustrate a second exemplary implantation system 700 in accordance with the present disclosure, in a ready-to-deploy state. Compared to the first implantation system 300 shown in FIGS. 3A-3B, the second implantation system 700 may facilitate attachment between the tag and the anti-migration device during the implantation procedure due to the use of a multi-part sheath construction.

The implantation system 700 comprises a handle portion 702 and a cannula 704 within which a deployment package 714 is disposed. The handle portion 702 comprises a deployment button 708 which operates a plunger 710, which in turn operates a driving element 712. The handle portion 702 and one or more components thereof may be produced from a suitable plastic. The deployment button 708 is connected to a housing of the handle portion 702 and is actuatable by a user, such as a surgeon. The deployment button 708 may physically be depressed or slid or may be a soft button configured to generate an electric or electronic signal upon actuation. The cannula 704 extends a distance away from the handle portion 702 and terminates in a cannula tip 706. The cannula 704 may be formed from a suitable metal. The cannula 704 has a length which may range from 25 mm (1 in.) to 200 mm (8 in.), and in example embodiments may range between 50 mm (2 in.) and 150 mm (6 in.). The length of the cannula 704 affects the range of the implantation system 700 in respect of the reachability of tissue sites in the body of a subject. A longer-length cannula 704 may be used when adjustment aids are used, for example for stereotaxis. The cannula 704 terminates in a cannula tip 706. The inner diameter of the cannula 704 may closely match the outer diameter of the deployment package 714 and may be approximately 0.15 mm (0.006 in.). For purposes of explanation, the direction of the cannula tip 706 is referred to herein as the “distal” direction and the direction of the handle portion 702 is referred to herein as the “proximal” direction.

The driving element 712 extends through the cannula 704. When the deployment button 708 is actuated, the plunger 710 operates to cause the driving element 712 to move in an axial direction within the cannula 704. The driving element 701 may be formed of a wire or a sufficiently stable plastic fiber. Consequently, the driving element 712 may be moved along a straight, guided sliding path between a pre-loaded position and a deployment position. In the deployment position, force from the plunger 710 is transferred to the deployment package 714 which has been pre-loaded at a location near to the cannula tip 706. Thus, when the deployment button 708 is actuated, the deployment package 714 may be driven out of the cannula 704 to the tissue site at the location of the cannula tip 706 by the axial movement of the driving element 712. This may be achieved by virtue of the driving element 712 being aligned coaxially with respect to the cannula 704 and hence pushing the pre-loaded deployment package 714 out of the cannula 704 past the cannula tip 706. One example of the deployment package 714 is shown in more detail in inset B, corresponding to FIG. 7B.

In FIG. 7B, the driving element 712 is in the pre-loaded position. The deployment package 714 includes a PIT tag 716 distal to the driving element 712 and an anti-migration device divided into a first anti-migration device portion 718 distal to the driving element 712 and proximal to the cannula tip 706, and a second anti-migration device portion 720 distal to the driving element 712 and proximal to the PIT tag 716. The PIT tag 716 may be, for example, the PIT tag 100 illustrated in FIG. 1. As an alternative to the configuration shown in FIG. 7B, the deployment package 714 of FIG. 7A may be formed of a PIT tag 716 that is already disposed in anti-migration device 718, as described in more detail below with regard to FIGS. 11A-11B. By comparison, however, the configuration shown in FIG. 7B may permit the use of a cannula having a smaller diameter. The anti-migration device may have the same overall form factor as, for example, any of the anti-migration devices 200a-200h illustrated in FIGS. 2A-2H or a modification or alternative thereto as described above, except that the anti-migration device of FIG. 7B is divided into the two portions. The first and second anti-migration devices 718, 720 are in a compressed state thereof. On account of their pre-stress, the first and second anti-migration devices 718, 720 (and thus the PIT tag 716) maintain their position within the cannula 304.

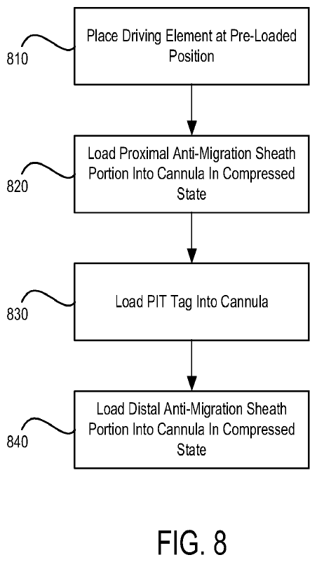

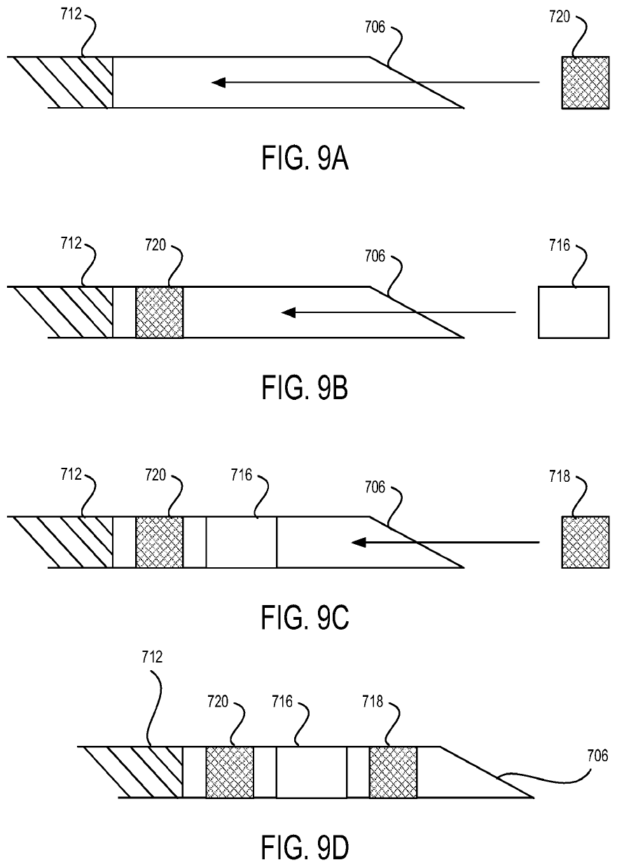

FIG. 8 illustrates an exemplary process flow for pre-loading the deployment package 714 in the deployment system 700. For purposes of explanation, the process flow will be described in the context of the deployment system 700 illustrated in FIG. 7. In this illustration, various processes within the process flow are illustrated in FIGS. 9A-9D. At operation 810, the process flow includes placing the driving element 712 at the pre-loaded position. Operation 810 may include actuating a button, such as the deployment button 708 or a reset/return button (not illustrated). In the event that the driving element 712 is already at the pre-loaded position, operation 810 may be omitted. Thereafter, at operation 820, the second anti-migration device portion 720 (being the portion that will be located proximal to the driving element 712) is loaded into the cannula 704, for example via the cannula tip 706. An example of operation 820 is illustrated in FIG. 9A. The second anti-migration device portion 720 may be loaded such that a certain distance exists between the second anti-migration device portion 720 and the driving element 712. If the proximal end of the cannula 304 is accessible (e.g., before a portion of the handle portion 302 is attached), one or more of the PIT tag 716, the first anti-migration device portion 718, and/or the second anti-migration device portion 720 may be loaded via the proximal end.

Next, at operation 830, the PIT tag 716 is loaded into the cannula 704, for example via the cannula tip 706. An example of operation 830 is illustrated in FIG. 9B. The PIT tag 716 may be loaded such that a certain distance exists between the PIT tag 716 and the second anti-migration device portion 720. After operation 830, at operation 840 the first anti-migration device portion 718 (being the portion that will be located proximal to the cannula tip 706) is loaded into the cannula 704, for example via the cannula tip 706. An example of operation 840 is illustrated in FIG. 9C. The first anti-migration device portion 718 may be loaded such that a certain distance exists between the first anti-migration device portion 718 and the PIT tag 716. After operation 840, the deployment system 700 is in the deployment-ready state as illustrated in FIG. 9D. It can be seen that the deployment-ready state illustrated in FIG. 9D corresponds to the inset of FIG. 7B.

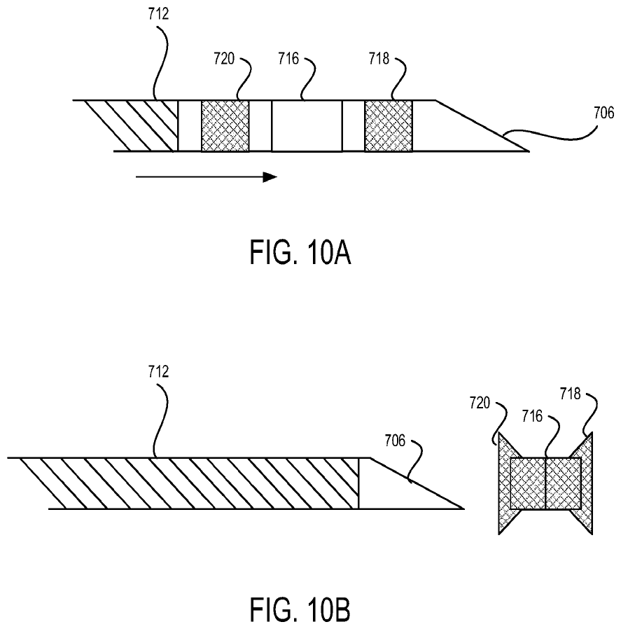

The deployment system 700 may exist in the deployment-ready state for an extended period of time (e.g., years) until the deployment package 714 is to be used. At such time, the deployment system 700 may be operated to simultaneously (or nearly simultaneously) deploy the entire deployment package 714. FIGS. 10A-10B illustrate the deployment procedure of the deployment system 700. Initially, as shown in FIG. 10A, the deployment system 700 is provided in the deployment-ready state. This state also corresponds to the state shown in FIGS. 7B and 9D. Subsequently, the operator (e.g., a surgeon) actuates the deployment button 708 causing the driving element 712 to move from the pre-loaded position to a deployment position. In this position, as shown in FIG. 10B, the driving element 712 has been extended to a point near the cannula tip 706. The contact force of the driving element 712 on the second anti-migration device portion 720, and thus on the PIT tag 716 thus on the first anti-migration device portion 718 causes the deployment package 714 to exit the cannula 704 as a sheathed PIT tag, in which the PIT tag 716 resides within the first and second anti-migration device portions 718, 720 and in which the overall anti-migration device has returned to its flared shape. For illustration purposes, the deployed anti-migration device is illustrated as having a longer length than the PIT tag 716, but in practical implementations the combined length of the first and second anti-migration device portions 718, 720 may preferably be shorter or of equal length compared to the PIT tag 716, such that it does not extend beyond the PIT tag 716 in either direction.

While FIGS. 10A-10B illustrate a deployment procedure in which the cannula tip 706 remains generally stationary relative to the deployment area while the driving element 712 and deployment package 714 move into the deployment area, in some implementations the converse may be true. In such implementations, the cannula 704 with the deployment package 714 may be advanced to the deployment site and, subsequently, the cannula 704 may be withdrawn (e.g., over a guidewire or the driving element 712). When the cannula 704 becomes sufficiently retracted that the cannula tip 706 clears the deployment package 714, the deployment package 314 will have been deployed into the deployment area. The cannula 704 may subsequently be fully withdrawn. The retraction of the cannula tip 706 may be automatically initiated in response to the actuation of the deployment button 708, or the operator may manually retract the cannula tip 706. Such a procedure may be beneficial in cases where hard tissue (e.g., a tumor) is located at the deployment area, as such a procedure may reduce lateral migration that would otherwise be caused by the force of the deployment package 714 against the hard tissue. In such a case, the cannula tip 706 itself may initially be used to puncture the hard tissue and thereby create a secure deposition location for the deployment package 714.

Generally, the PIT tag 716 may enter the first and second anti-migration device portions 718, 720 in one of two ways, depending on the relative physical parameters (e.g., relative diameters) of the cannula 704, the PIT tag 716, and the first and second anti-migration device portions 718, 720; and in some cases, on the relative stiffness of the tissue into which the deployment package 314 is deployed. In the first instance, the force of the driving element 712 pushes the compress second anti-migration device portion 720 over the PIT tag 716 and pushes the PIT tag 716 into the compressed first anti-migration device portion 718 within the cannula 704, such that the anti-migration device securely holds the PIT tag 716. The force of the driving element 712 may then subsequently push both the PIT tag 716 and the first and second anti-migration device portions 718, 720 into the tissue simultaneously. In the second instance, the force of the driving element 712 causes the second anti-migration device portion 720 itself to exert a force on the PIT tag 716 and on the first anti-migration device portion 718, causing the first anti-migration device portion 718 to enter the tissue and expand. The driving element 712 continues to exert a force on the second anti-migration device portion 720 to drive the PIT tag 716 out of the cannula 704 and partially into the expanded first anti-migration device portion 718, and finally to drive the second anti-migration device portion 720 out of the cannula and over the exposed portion of the PIT tag 716. As such, the PIT tag 716 becomes encapsulated and securely held by the anti-migration device within the tissue.

In any of the above procedures, the deployment procedure may be guided through the use of an imaging technology such as, for example, ultrasound, X-ray, and/or MRI imaging modalities. For example, an operator may monitor the implantation area to ensure that the PIT tag 716 and the first and second anti-migration portions 718, 720 are properly positioned with respect to one another and with respect to the implantation area itself. Thus, the operator may ensure that the combined deployment package 714, upon deployment, is appropriately situated relative to the lesion, tumor, or other object whose location the PIT tag 716 is intended to mark.

While in FIGS. 3A-3B and 7A-7B, the deployment package 314, 714 is illustrated as being composed of several components axially displaced from one another, in alternative implementations the PIT tag and the anti-migration device may be assembled before loading into the deployment apparatus. In such implementations, the anti-migration device may be fixed to the PIT tag by a spring force, over molding, shrink-tubing, a glue joint, a friction fit, a form fit, solvent bonding and the like. An example of such an implementation is illustrated in FIGS. 11A-11B. Where the implantable shell of the PIT tag is of a two-part construction (e.g., as illustrated in FIG. 1), the anti-migration device may be fixed by partially inserting the anti-migration device between the two parts. Utilizing the deployment methods illustrated in FIGS. 3A-3B or 7A-7B may reduce the likelihood of a stuck deployment, in which the deployment package fails to properly exit the cannula. Moreover, the deployment methods illustrated in FIGS. 3A-3B or 7A-7B may permit the use of a cannula with a reduced diameter, for example because the deployment package itself may have a reduced diameter. Comparatively, the deployment systems illustrated in FIGS. 11A-11B may permit a wider range of usable designs for the anti-migration device.

FIGS. 11A-11B illustrate a third exemplary implantation system 1100 in accordance with the present disclosure, in a ready-to-deploy state. The implantation system 1100 comprises a handle portion 1102 and a cannula 1104 within which a deployment package 1114 is disposed.

The handle portion 1102 comprises a deployment button 1108 or other actuator which operates a plunger 1110, which in turn operates a driving element 1112. The handle portion 1102 and one or more components thereof may be produced from a suitable plastic. The deployment button 1108 is connected to a housing of the handle portion 1102 and is actuatable by a user, such as a surgeon. The deployment button 1108 may physically be depressed or moved or may be a soft button configured to generate an electric or electronic signal upon actuation. The cannula 1104 extends a distance away from the handle portion 1102 and terminates in a cannula tip 1106. The cannula 1104 may be formed from a suitable metal. The cannula 1104 has a length which may range from 25 mm (1 in.) to 200 mm (8 in.), and in some example embodiments is between 50 (2 in.) mm and 150 mm (6 in.). The length of the cannula 1104 affects the range of the implantation system 1100 in respect of the reachability of tissue sites in the body of a subject. A longer-length cannula 1104 may be used when adjustment aids are used, for example for stereotaxis. The cannula 1104 terminates in a cannula tip 1106. The inner diameter of the cannula 1104 may closely match the outer diameter of the deployment package 1114 and may be approximately 0.15 mm (0.006 in.). For purposes of explanation, the direction of the cannula tip 1106 is referred to herein as the “distal” direction and the direction of the handle portion 1102 is referred to herein as the “proximal” direction.

The driving element 1112 extends through the cannula 1104. When the deployment button 1108 is actuated, the plunger 1110 operates to cause the driving element 1112 to move in an axial direction within the cannula 1104. The driving element 1112 may be formed of a wire or a sufficiently stable plastic fiber. Consequently, the driving element 1112 may be moved along a straight, guided sliding path between a pre-loaded position and a deployment position. In the deployment position, force from the plunger 1110 is transferred to the deployment package 1114 which has been pre-loaded at a location near to the cannula tip 1106. Thus, when the deployment button 1108 is actuated, the deployment package 1114 may be driven out of the cannula 1104 to the tissue site at the location of the cannula tip 1106 by the axial movement of the driving element 1112. This may be achieved by virtue of the driving element 1112 being aligned coaxially with respect to the cannula 1104 and hence pushing the pre-loaded deployment package 1114 out of the cannula 1104 past the cannula tip 1106. One example of the deployment package 1114 is shown in more detail in inset B, corresponding to FIG. 11B.

In FIG. 11B, the driving element 1112 is in the pre-loaded position. The deployment package 1114 is pre-assembled and includes a PIT tag 1116 surrounded by an anti-migration device 1118, both of which are distal to the driving element 1112 and proximal to the cannula tip 1106. While FIG. 11B illustrates the PIT tag 1116 as being wholly encapsulated by the anti-migration device 1118, in some implementations only a portion (e.g., only the distal portion or only the proximal portion) of the PIT tag 1116 may be so encapsulated. The PIT tag 1116 may be, for example, the PIT tag 100 illustrated in FIG. 1. The anti-migration device 1118 may be, for example, any of the anti-migration devices 200a-200h illustrated in FIGS. 2A-2H or may be a modification or alternative thereto as described above. The anti-migration device 1118 is in a compressed state. On account of pre-stress, the anti-migration device 1118 (and thus the PIT tag 1116) maintains its position within the cannula 1104.

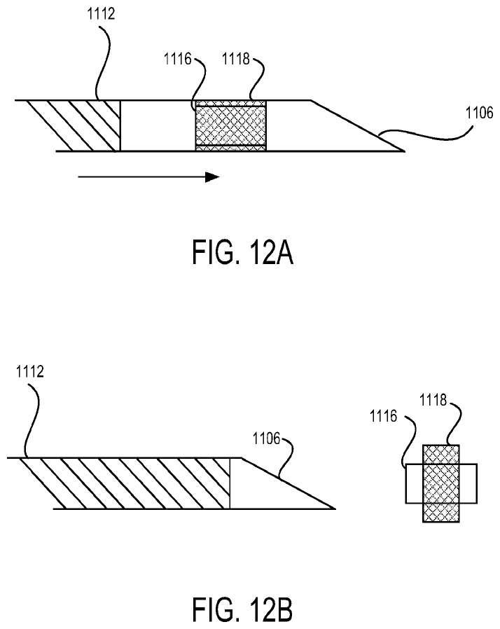

Compared to the processes for pre-loading the deployment packages 314, 714 of FIGS. 3A-3B and 7A-7B, which are described above with regard to FIGS. 4 and 8, a process flow for pre-loading the deployment package 1114 of FIG. 11B may be relatively simplified. For example, one may place the driving element 1112 at the pre-loaded position and subsequently loading the pre-assembled deployment package 1114 comprising the PIT tag 1116 and the anti-migration device 1118, either via the cannula tip 1106 or, if the proximal end of the cannula 1104 is accessible, via the proximal end. After loading the pre-assembled deployment package 1114, the deployment system 1100 is in the deployment-ready state which corresponds to the inset of FIG. 11B. The deployment system 1100 may exist in the deployment-ready state for an extended period of time (e.g., years) until the deployment package 1114 is to be used. At such time, the deployment system 1100 may be operated to deploy the pre-assembled deployment package 1114. FIGS. 12A-12B and 13A-13B illustrate the deployment procedure of the deployment system 1100 for two different examples of the pre-assembled deployment package 1114.

Initially, as shown in FIGS. 12A and 13A, the deployment system 1100 is provided in the deployment-ready state. In FIG. 12A, the PIT tag 1116 is substantially entirely encapsulated within the anti-migration device 1118, which may be implemented as a cage-like or basket-like resilient device. In FIG. 13A, however, only the proximal portion of the PIT tag 1116 is encapsulated within the anti-migration device 1118, which may be implemented as a semi-hourglass or trumpet-like resilient device. Subsequently, the operator (e.g., a surgeon) actuates the deployment button 1108 causing the driving element 1112 to move from the pre-loaded position to a deployment position. In this position, as shown in FIGS. 12B and 13B, the driving element 112 has been extended to a point near the cannula tip 1106. The contact force of the driving element 1112 on the deployment package 1114 causes the deployment package 1114 to exit the cannula 1104. As shown in FIG. 12B, after deployment the anti-migration device 1118 holds the PIT tag 1116 securely within at approximately the center (in an axial direction) of the PIT tag 1116, while simultaneously the outer portion of the anti-migration device 1118 has expanded radially outward to resist subsequent migration of the PIT tag 1116. As shown in FIG. 13B, after deployment a distal portion of the anti-migration device 1118 securely holds the proximal portion of the PIT tag 1116, while a proximal portion of the anti-migration device 1118 has expanded radially outward such that the anti-migration device 1118 has a flared or trumpet-like shape. Thus, the anti-migration device 1118 resists subsequent migration of the PIT tag 1116, and may be especially resistant to migration along the channel created by the cannula 1104 after the cannula 1104 is removed. While FIG. 13B illustrates the anti-migration device 1118 being engaged with the proximal portion of the PIT tag 1116, in other implementations the proximal portion of the anti-migration device 1118 may instead be engaged with the distal portion of the PIT tag 1116 with the distal portion of the anti-migration device 1118 expanding radially outward with the flared or trumpet-like shape.

FIGS. 14A-14L and 140-14T respectively illustrate examples of the deployed packages. For ease of illustration and explanation, the deployed packages are of a unitary anti-migration device construction and thus deployed by the deployment system 300 illustrated in FIGS. 3A-3B. However, the packages may have a similar appearance even if a two-part construction is used for the anti-migration device construction. To further illustrate the wide range of potential designs for the anti-migration device, the particular designs shown in FIGS. 14A-14L and 140-14T are not selected from those expressly illustrated in FIGS. 2A-2J but rather are further examples of the modified sheaths described above. Moreover, FIGS. 14E-14L show examples in which an anti-migration device is used concurrently with a cap-like inner anti-migration device, such as the anti-migration device 200h illustrated in FIG. 2H. However, the examples illustrated in FIGS. 14E-14L may be used without the cap-like inner anti-migration device. Moreover, in each of FIG. 14E-14L and 140-14T, the anti-migration devices may be formed of at least one nitinol wire which is wrapped around the PIT tag.

In particular, FIG. 14A illustrates the expanded form of a PIT tag 1410a encapsulated in an anti-migration device 1420a that has a generally flower-like construction in the axial direction, including a plurality of petal-like flanged portions flared at the axial extremes thereof (which may be considered as a type of protrusion in accordance with the present disclosure), with a central portion to hold the PIT tag 1410a. FIG. 14B illustrates the expanded form of a PIT tag 1410b encapsulated in an anti-migration device 1420b that is similarly flower-like, but formed of a thicker material. FIG. 14C the compressed form of illustrates a PIT tag 1410c that is nearly entirely enveloped in a spiral-shaped anti-migration device 1420c. The grooves between adjacent portions of the spiral may provide small-diameter regions; however, in other implementations the grooves may extend through the entirety of the anti-migration device 1420c such that gaps exist between adjacent portions of the spiral. In the expanded form, the diameter of the anti-migration device 1420c may be, for example, two to five times larger than in the compressed form. As noted above, in such an implementation in may be preferable to form the anti-migration device 1420c from a material that is substantially permeable to RF radiation. FIG. 14D illustrates the expanded form of a PIT tag 1410d that is contained within a thin spiral-shaped anti-migration device 1420d which includes protruding ends. In such an implementation, the protruding ends may be formed in any shape which provides anti-migration properties to the deployed package.

As noted above, anti-migration devices in accordance with the present disclosure need not exhibit rotational symmetry. Additionally, if an anti-migration device includes a flared or protruding portion, said portion is not necessarily located at the end(s) of the anti-migration device. For example, FIG. 14E illustrates the expanded form of a PIT tag 1410e encapsulated in an anti-migration device 1420e which has a generally spiral-or coil-shaped main portion, with two radially-outward protruding portions (in which the coil diameter is increased) which are axially displaced away from the ends of the anti-migration device 1420e and toward a center portion of the anti-migration device 1420e. In the compressed form, the wires may not overlap in order to prevent the need for a larger cannula diameter. While FIG. 14E illustrates two protruding portions and shows both portions protruding in the same radial direction, in other examples the anti-migration device may include one protruding portion or more than two protruding portions, and the protruding portions may protrude in different radial directions. In some implementations, the protruding portions may be capable of twisting in one or more directions (e.g., in a tangential direction and/or an axial direction of the PIT tag 1410e).

In FIG. 14F, a PIT tag 1410f is encapsulated in an anti-migration device 1420f which has a generally spiral-or coil-shaped main portion with two sets of radially protruding portions, one set at each end of the anti-migration device 1420f, shown in the expanded form. As noted above, any number of radially protruding portions, sets of radially protruding portions, or other various combinations of radially protruding portions (e.g., one protruding portion or more than two protruding portions) may be present at a given end, and the number of radially protruding portions at one end of the sheath may be different than the number of radially protruding portions at the opposite end of the sheath. In some implementations, only one end may have any radially protruding portions. One such example is illustrated in the expanded form shown in FIG. 14G, in which a PIT tag 1410g is encapsulated in an anti-migration device 1420g which has a generally spiral-or coil-shaped main portion with two radially protruding portions at only one end of the anti-migration device 1420g. In any implementation, the radially protruding portions may be capable of twisting in one or more directions. In FIG. 14G, one example of a position to which the radially protruding portions may twist is illustrated with a dashed line, in which the radially protruding portions rotate between an axial forward direction and an axial rearward direction of the sheath.

In other examples, as shown in FIGS. 140-14T , a PIT tag 14100, 1410q, 1410s is encapsulated in an anti-migration device 14200, 1420q, 1420s, which has a generally spiral or coil-shaped radially expanded and protruding main portion (i.e., which form the anti-migration portion of the device 14200, 1420q, 1420s), with two spiral or coil-shaped radially fixed end portions that grip the rounded ends of the PIT tag 14100, 1410q, 1410s. As discussed above, the anti-migration device 14200, 1420q, 1420s can be formed from one or more wires (e.g., one or more wires formed from a shape-memory material, such as, for example, nitinol). FIGS. 140 and 14P, for example, illustrate an anti-migration device 14200 that is formed from two nitinol wires. FIGS. 14Q and 14R illustrate an anti-migration device 1420q that is formed from three nitinol wires. And FIGS. 14S and 14T illustrate an anti-migration device 1420s that is formed from four nitinol wires. As illustrated in FIGS. 140-14T, in the expanded form, the anti-migration device 14200, 1420q, 1420s has a generally flower-like construction in the axial direction, which includes a main portion with a plurality of petal-like flanged portions that are flared radially (i.e., extending in different radial directions from the marker body, when viewed in an axial direction, and which may be considered as a type of protrusion in accordance with the present disclosure), with the fixed end portions holding the rounded ends of the PIT tag 14100, 1410q, 1410s. Anti-migration devices in accordance with the present disclosure may, however, employ any number of wires, formed from various materials, and spiraling around the PIT tag in various configurations, as would be understood by those of ordinary skill in the art.

In other examples, an anti-migration device in accordance with the present disclosure may be implemented as a cage-type matrix which entirely surrounds the PIT tag, thereby to define an interior in which the PIT tag is disposed. In FIG. 14H, a PIT tag 1410h is entirely enclosed within a cage-type anti-migration device 1420h. The cage-type anti-migration device 1420h may be attached to the PIT tag 1420h, for example by one or more wires, anchors, or coils, thereby to reduce or prevent movement of the PIT tag 1410h within the cage-type anti-migration device 1420h. The cage-type anti-migration device 1420h may expand upon deployment to move away from the PIT tag 1410h and define an area from which the PIT tag 1410h is contained in due to anchoring of the cage within the tissue and the PIT tag 1410h may be free to move within the area defined by the cage-type anti-migration device 1420h. In any implementation, the cage-type anti-migration device 1420h may be anchored by the cage-type structure or sheath 1420h may include additional structures that act as anchors such as the radially-extending protruding portions described with regard to other examples herein. When using the cage-type anti-migration device 1420h, the material may be selected such that the sheath does not act as a Faraday cage and instead permits RF and/or other low frequency electromagnetic radiation to pass therethrough to the PIT tag 1410h.

Anti-migration devices in accordance with the present disclosure do not necessarily completely surround the PIT tags in a circumferential direction, but instead may be comprised of multiple separate portions in the circumferential direction. In such implementations, the anti-migration devices may be attached to the PIT tags (e.g., by welding) prior to insertion in a deployment system. FIGS. 14I and 14J illustrate the expanded form of two such examples. In FIG. 14I, a PIT tag 1410i is affixed with four anti-migration flaps 1420i which collectively form the anti-migration device, although only three of the anti-migration flaps 1420i are visible in the side view of FIG. 14I. The anti-migration flaps 1420i may instead be wire portions (i.e., may have a narrower width than illustrated in FIG. 14L), and may be formed of any one or more of the materials described above. Each of the anti-migration flaps 1420i is curved away from the PIT tag 1410i at the end thereof in the expanded form, such that they cut into the tissue when the PIT tag 1410j is deployed. In FIG. 14J, a PIT tag 1410j is affixed with two anti-migration flaps 1420j which collectively form the anti-migration device. As opposed to the welded or cemented configuration shown in FIG. 14I, the anti-migration flaps 1420j of FIG. 14J are inserted between the two portions which make up the shell of the PIT tag 1410j. In practical implementations of the examples shown in FIGS. 14I and 14J, any number of flaps may be used. The flaps may be formed of, for example, nitinol having a thickness of 0.05 mm (0.002 in.) to 0.2 mm (0.08 in.).

In addition to or instead of the protrusions, flaps, or wires described above, an anti-migration device in accordance with the present disclosure may include other types of anti-migration features. In FIG. 14K, a PIT tag 1410k is partially encapsulated in a torsion coil anti-migration device 1420k, shown in the expanded form. The torsion coil anti-migration device 1420k may be affixed to the PIT tag 1410k near a center of the PIT tag 1410k such that the portion of the torsion coil anti-migration device 1420k near the end of the PIT tag 1410k is free. Upon deployment, the free end may expand within the tissue so as to provide an outward radial force to anchor the PIT tag 1410k. In FIG. 14L, a PIT tag 14101 is partially encapsulated in an anti-migration device 14201 which terminates in a barbed or serrated cone. The barbs or serrations may resist movement of the PIT tag 14101 in the longitudinal direction. Such an implementation may be used if a practitioner would like to anchor the PIT tag 14101 to a particular feature (e.g., a mass within the tissue). The barbs and/or serrations may also be combined with protruding portions, either as separate features or as a combined feature (e.g., a fishhook-like portion).

An anti-migration device in accordance with the present disclosure may also include one or more features to aid with the deployment process by receiving a force from the driving element. In implementations where an anchor-type anti-migration feature is used (such as that shown in FIG. 14L), sharpened feature may improve the ability of the anti-migration device to cut into harder tissue (e.g., tumor tissue) while reducing lateral movement during implantation. One such example is illustrated in FIG. 14M, which shows two perspective views of a PIT tag 1410m that is encapsulated in an anti-migration device 1420m. At the distal end of the anti-migration device 1420m, a spade-shaped anti-migration feature is provided. This anti-migration feature may assist in cutting into the tissue. At the proximal end of the anti-migration device 1420m, a seat feature is provided which partially curves around the proximal end of the PIT tag 1410m. The seat feature may directly receive force from the driving element, thus increasing the force with which the anti-migration device 1420m may enter the tissue (e.g., by increasing the force applied by the spade-shaped anti-migration feature). A seat feature, such as that shown in FIGS. 14M and 14N, may be used with any of the anti-migration devices illustrated and/or described above, and is not limited to use with anti-migration devices having spade-type anti-migration features.

Illustrative examples of the marking systems, deployment systems, methods of marking a target location, and localization marker devices are provided below. Embodiments of the systems, methods, and devices described herein may include any one or more, and any combination of, the clauses described below:
Clause 1. A marking system for implantation into tissue, comprising: a marker body having an outer surface, the marker body including: a transponder configured to respond to electromagnetic radiation from an external source, and a microchip operatively connected to the transponder and configured to activate in response to the electromagnetic radiation; and an anti-migration device configured to receive at least a portion of the marker body and to engage tissue in which the marker body is implanted to resist movement of the marker body after implantation.
Clause 2. The system according to clause 1, wherein the anti-migration device includes a portion configured to transition between a compressed state and an expanded state.
Clause 3. The system according to clause 2, wherein the anti-migration device has a first size and a first shape in the compressed state and the anti-migration device has a second size and/or a second shape in the expanded state, wherein one or both the second size and the second shape are different than the first size and the first shape, respectively.
Clause 4. The system according to clause 3, wherein a portion of the anti-migration device is configured to expand from the first size to the second size during implantation of the marking system.
Clause 5. The system according to clause 4, wherein the portion of the anti-migration device configured to expand from the first size to the second size is an end portion of the anti-migration device.
Clause 6. The system according to clause 4 or clause 5, wherein the portion of the anti-migration device configured to expand from the first size to the second size includes first and second end portions of the anti-migration device.
Clause 7. The system according to any one of clauses 4-6, wherein the portion of the anti-migration device configured to expand from the first size to the second size is formed of a braided, woven, mesh, or webbed structure.
Clause 8. The system according to clause 4, wherein the portion of the anti-migration device configured to expand from the first size to the second size is located at a central portion of the anti-migration device.
Clause 9. The system according to clause 8, wherein the portion of the anti-migration device configured to expand from the first size to the second size is formed of a braided, woven, mesh, or webbed structure.
Clause 10. The system according to any one of clauses 3-9, wherein a portion of the anti-migration device is configured to change from the first shape to the second shape during implantation of the marking system.
Clause 11. The system according to clause 10, wherein the portion of the anti-migration device configured to change shape includes at least one protrusion.
Clause 12. The system according to clause 10 or clause 11, wherein the portion of the anti-migration device configured to change shape includes a plurality of protrusions.
Clause 13. The system according to clause 11 or clause 12, wherein the at least one protrusion is configured to move from an axially extending position to a radially extending position during implantation of the marking system.
Clause 14. The system according to any one of clauses 11-13, wherein the at least one protrusion includes a point configured to engage tissue in which the body is implanted to resist movement of the marking system.
Clause 15. The system according to any one of clauses 1-14, wherein the anti-migration device includes a hollow structure configured to receive a portion of the marker body and wherein an end of the hollow structure includes a sharpened point configured to penetrate hard or bony tissue.
Clause 16. The system according to any one of clauses 1-15, wherein the anti-migration device is at least partially formed of a shape memory material.
Clause 17. The system according to clause 16, wherein the shape memory material is at least one of silicone, ethylene propylene diene monomer, thermoplastic elastomer, polyether ether ketone, polypropylene, or combinations thereof.
Clause 18. The system according to clause 16, wherein the material is nitinol.
Clause 19. The system according to any one of clauses 1-18, wherein the anti-migration device comprises a sheath configured to receive at least a portion of the marker body.
Clause 20. The system according to clause 19, wherein the sheath has a unitary construction.
Clause 21. The system according to clause 19, wherein the sheath includes a first sheath portion and a second sheath portion.
Clause 22. The system according to any one of clauses 19-21, wherein the sheath is formed of a braided, woven, mesh, or webbed structure.
Clause 23. The system according to clause 22, wherein the braided, woven, mesh, or webbed structure has an hourglass shape.
Clause 24. The system according to clause 22 or clause 23, wherein the braided, woven, mesh, or webbed structure defines an interior in which the marker body is disposed.
Clause 25. The system according to any one of clauses 22-24, wherein the braided, woven, mesh, or webbed structure is configured to engage a distal portion of the marker body or a proximal portion of the marker body.
Clause 26. The system according to any one of clauses 19-25, wherein at least a portion of the sheath is configured to radially expand away from the outer surface of the marker body during implantation.
Clause 27. The system according to clause 1, wherein the anti-migration device includes a portion configured to transition between a tensioned state and an expanded state.
Clause 28. The system according to clause 1, wherein the anti-migration device is formed from one or more wires, the one or more wires forming a spiral body comprising a radially expanded and protruding main portion and two radially fixed end portions, the radially fixed end portions of the spiral body being configured to grip the marker body.
Clause 29. A deployment system, comprising: a deployment device, including: a handle portion including an actuator configured to cause a deployment operation, and a cannula attached to the handle portion at a first end of the cannula, wherein the cannula terminates in a cannula end at a second end of the cannula opposite the first end; and a marking system disposed within a lumen of the cannula, the marking system including: a marker body having an outer surface, the marker body including: a transponder configured to respond to electromagnetic radiation from an external source, and a microchip operatively connected to the transponder and configured to activate in response to the electromagnetic radiation, and an anti-migration device configured to receive at least a portion of the marker body, the anti-migration device being configured to resist movement of the marker body after implantation of the marking system in tissue.
Clause 30. The deployment system according to clause 29, wherein the marker body is at least partially disposed within the anti-migration device while disposed within the lumen of the cannula.
Clause 31. The deployment system according to clause 29, wherein the anti-migration device is axially displaced from the marker body within the lumen of the cannula.
Clause 32. The deployment system according to clause 29, wherein a first portion of the anti-migration device is axially displaced from the marker body in a first direction in the lumen of the cannula, and a second portion of the anti-migration device is axially displaced from the marker body in a second direction opposite the first direction in the lumen of the cannula.
Clause 33. The deployment system according to any one of clauses 29-32, wherein the deployment device further includes a lubricant configured to facilitate relative movement between the marking system and the lumen of the cannula.
Clause 34. A method of marking a target location in a patient's body, the method comprising: positioning a distal end of a cannula adjacent to the target and underneath a tissue surface, wherein a tissue marker and an anti-migration device are disposed in a lumen of the cannula; deploying the tissue marker and the anti-migration device at the target location; and expanding a portion of the anti-migration device to engage tissue at the target location to anchor the tissue marker at the target location.
Clause 35. The method according to clause 34, wherein expanding a portion of the anti-migration device to engage tissue at the target location includes releasing at least one wire portion of the anti-migration device from a compressed state and engaging the tissue at the target location with the at least one wire portion of the anti-migration device.
Clause 36. The method according to clause 34, wherein expanding a portion of the anti-migration device to engage tissue at the target location includes radially expanding a sheath portion of the anti-migration device from a compressed state and engaging the tissue at the target location with the radially expanded sheath portion of the anti-migration device.
Clause 37. The method according to any one of clauses 34-36, further comprising positioning at least a portion of the tissue marker within an interior portion of the anti-migration device.
Clause 38. The method according to clause 37, wherein positioning at least a portion of the tissue marker within the interior portion of the anti-migration device occurs after expanding a portion of the anti-migration device to engage tissue at the target location.
Clause 39. The method according to clause 37, wherein positioning at least a portion of the tissue marker within the interior portion of the anti-migration device occurs prior to expanding a portion of the anti-migration device to engage tissue at the target location.
Clause 40. The method according to any one of clauses 34-39, wherein the anti-migration device is in a compressed state when disposed in the lumen of the cannula.
Clause 41. The method according to any one of clauses 34-40, further comprising: prior to positioning the distal end of the cannula, pre-assembling the tissue marker and the anti-migration device into a deployment package.
Clause 42. The method according to clause 41, wherein pre-assembling the tissue marker and the anti-migration device into a deployment package includes attaching the anti-migration device to the tissue marker by at least one of a spring force, an overmolding process, a shrink-tubing process, a glue joint, a friction fit, a form fit, or a solvent bonding process.
Clause 43. The method according to any one of clauses 34-40, wherein deploying the tissue marker and the anti-migration device comprises: deploying the anti-migration device at the target location; and subsequently, deploying the tissue marker at the target location.
Clause 44. The method according to any one of clauses 34-40 or 43, wherein deploying the tissue marker at the target location includes positioning a portion of the tissue marker in an interior of the anti-migration device subsequent to deploying the anti-migration device.
Clause 45. The method according to clause 44, wherein positioning at least a portion of the tissue marker within the interior of the anti-migration device occurs prior to expanding a portion of the anti-migration device to engage tissue at the target location.
Clause 46. The method according to clause 44, wherein positioning at least a portion of the tissue marker within the interior of the anti-migration device occurs after expanding a portion of the anti-migration device to engage tissue at the target location.
Clause 47. The method according to any one of clauses 34-40, wherein deploying the tissue marker and the anti-migration device comprises: deploying a first portion of the anti-migration device at the target location; subsequent to deploying the first portion of the anti-migration device, deploying the tissue marker at the target location; and subsequent to deploying the tissue marker, deploying a second portion of the anti-migration device at the target location.
Clause 48. The method according to clause 47, wherein deploying the tissue marker at the target location includes positioning a portion of the tissue marker in an interior of the first portion of the anti-migration device.
Clause 49. The method according to clause 47 or clause 48, wherein deploying the second portion of the second portion of the anti-migration device at the target location includes positioning a portion of the tissue marker in an interior of the second portion of the anti-migration device.
Clause 50. The method according to any one of clauses 47-49, wherein expanding a portion of the anti-migration device to engage tissue at the target location includes expanding the first portion of the anti-migration device prior to deploying the tissue marker.
Clause 51. The method according to any one of clauses 47-50, wherein expanding a portion of the anti-migration device to engage tissue at the target location further includes expanding the second portion of the anti-migration device subsequent to deploying the tissue marker.
Clause 52. The method according to any one of clauses 34-51, wherein the tissue marker is a passive integrated transponder (PIT) tag.
Clause 53. A localization marker device for implantation into tissue, comprising: an implantable marker having a body with an outer surface, wherein the implantable marker is a transponder, a reflector, an active marker, a magnetic marker, a radioactive seed, a doppler marker, a passive marker, a wireless tumor location implant, or a combination thereof, and an anti-migration device configured to receive at least a portion of the body of the marker and to at least partially engage the outer surface of the body of the marker, the anti-migration device including a portion configured to transition between a first state and a second state, wherein in the first state the anti-migration device is configured to be disposed within a lumen of a cannula and in the second state the anti-migration device is configured to provide a movement resistance to the implantable marker after deployment from the cannula.
Clause 54. The device according to clause 53, wherein the anti-migration device includes at least one surface feature configured to increase the movement resistance.
Clause 55. The device according to clause 53 or clause 54, wherein the anti-migration device includes a first anti-migration device portion and a second anti-migration device portion.
Clause 56. The device according to clause 55, wherein the first anti-migration device portion is axially displaceable from the implantable marker in a first direction, and the second anti-migration device portion is axially displaceable from the implantable marker in a second direction opposite the first direction.
Clause 57. The device according to any one of clauses 53-56, wherein the anti-migration device has a mesh structure.
Clause 58. The device according to clause 57, wherein the mesh structure has an hourglass shape from a side-view perspective.
Clause 59. The device according to clause 57 or clause 58, wherein the mesh structure defines an interior in which the implantable marker is disposed.
Clause 60. The device according to any one of clauses 57-59, wherein the mesh structure is configured to engage one of a distal portion or a proximal portion of the implantable marker.
Clause 61. The device according to any one of clauses 57-60, wherein the mesh structure is a cage that surrounds the implantable marker.
Clause 62. The device according to clause 53, wherein the anti-migration device includes a nitinol wire with at least one barb.
Clause 63. The device according to clause 53, wherein the anti-migration device includes a tube with cut-out portions, the cut-out portions forming at least one barb.
Clause 64. The device according to clause 53, wherein the anti-migration device includes a spiral body formed from one or more wires.
Clause 65. The device according to clause 64, wherein when the anti-migration device is in the second state, the spiral body forms a plurality of flanged portions, the plurality of flanged portions extending in different radial directions from the marker body, when viewed in an axial direction.
Clause 66. The device according to clause 53, wherein in the first state the anti-migration device is compressed and in the second state the anti-migration device is expanded.
Clause 67. The device according to clause 53, wherein in the first state the anti-migration device is tensioned and in the second state the anti-migration device is expanded.

The marking system of any one of clauses 1-28 can be practiced with the localization marker device of any one of clauses 52-67 and/or in conjunction with the deployment system and/or method of marking a target location of any one of clauses 29-33 and/or 34-52.

The deployment system of any one of clauses 29-33 can be practiced with the marking system and/or the localization marker device of any one of clauses 1-28 and/or 53-67 and/or in conjunction with the method of marking a target location of any one of clauses 34-52.

The method of marking a target location of any one of clauses 34-52 can be practiced with the marking system of any one of clauses 1-28, the deployment system any one of clauses 29-33, and/or localization marker device of any one of clauses 53-67.

The localization marker device of any one of clauses 53-67 can be used with the marking system of any one of clauses 1-28, the deployment system any one of clauses 29-33, and/or the method of marking a target location of any one of clauses 34-52.

The above description and associated figures teach the best mode of implementing the devices and methods of the present disclosure and are intended to be illustrative and not restrictive. Many embodiments and applications other than the examples provided would be apparent to those skilled in the art upon reading the above description. The scope should be determined, not with reference to the above description, but instead with reference to the appended claims, along with the full scope of equivalents to which such claims are entitled. It is anticipated and intended that future developments will occur in the technologies discussed herein, and that the disclosed systems and methods will be incorporated into future embodiments. In sum, it should be understood that the application is capable of modification and variation.

Further, this description's terminology is not intended to limit the invention. For example, spatially relative terms-such as “beneath,” “below,” “lower,” “above,” “upper,” “proximal,” “distal,” and the like-may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

All terms used in the claims are intended to be given their broadest reasonable constructions and their ordinary meanings as understood by those knowledgeable in the technologies described herein unless an explicit indication to the contrary is made herein. In particular, the use of the singular articles such as “a,” “the,” “said,” etc. should be read to recite one or more of the indicated elements unless a claim recites an explicit limitation to the contrary.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A marking system for implantation into tissue, comprising:
a marker body having an outer surface, the marker body including:
a transponder configured to respond to electromagnetic radiation from an external source, and
a microchip operatively connected to the transponder and configured to activate in response to the electromagnetic radiation; and
an anti-migration device configured to receive at least a portion of the marker body and to engage tissue in which the marker body is implanted to resist movement of the marker body after implantation, wherein the anti-migration device includes a portion configured to transition between a compressed state and an expanded state.

2. (canceled)

3. The system according to claim 1, wherein the anti-migration device has a first size and a first shape in the compressed state and the anti-migration device has a second size and/or a second shape in the expanded state, wherein one or both the second size and the second shape are different than the first size and the first shape, respectively.

4. The system according to claim 3, wherein a portion of the anti-migration device is configured to expand from the first size to the second size during implantation of the marking system.

5. The system according to claim 4, wherein the portion of the anti-migration device configured to expand from the first size to the second size is an end portion of the anti-migration device.

6. (canceled)

7. The system according to claim 4, wherein the portion of the anti-migration device configured to expand from the first size to the second size is formed of a braided, woven, mesh, or webbed structure.

8. The system according to claim 4, wherein the portion of the anti-migration device configured to expand from the first size to the second size is located at a central portion of the anti-migration device.

9. The system according to claim 8, wherein the portion of the anti-migration device configured to expand from the first size to the second size is formed of a braided, woven, mesh, or webbed structure.

10. The system according to claim 3, wherein a portion of the anti-migration device is configured to change from the first shape to the second shape during implantation of the marking system.

11. The system according to claim 10, wherein the portion of the anti-migration device configured to change from the first to the second shape includes at least one protrusion.

12. (canceled)

13. The system according to claim 11, wherein the at least one protrusion is configured to move from an axially extending position to a radially extending position during implantation of the marking system.

14. The system according to claim 11, wherein the at least one protrusion includes a point configured to engage tissue in which the marker body is implanted to resist movement of the marking system.

15. The system according to claim 1, wherein the anti-migration device includes a hollow structure configured to receive a portion of the marker body and wherein an end of the hollow structure includes a sharpened point configured to penetrate hard or bony tissue.

16. The system according to claim 1, wherein the anti-migration device is at least partially formed of a shape memory material.

17. (canceled)

18. (canceled)

19. The system according to claim 1, wherein the anti-migration device comprises a sheath configured to receive at least a portion of the marker body.

20. The system according to claim 19, wherein the sheath has a unitary construction.

21. (canceled)

22. The system according to claim 19, wherein the sheath is formed of a braided, woven, mesh, or webbed structure.

23. The system according to claim 22, wherein the braided, woven, mesh, or webbed structure has an hourglass shape, defines an interior in which the marker body is disposed, and is configured to engage either a distal portion of the marker body or a proximal portion of the marker body.

24. (canceled)

25. (canceled)

26. The system according to claim 19, wherein at least a portion of the sheath is configured to radially expand away from the outer surface of the marker body during implantation.

27. A marking system for implantation into tissue, comprising:
a marker body having an outer surface, the marker body including:
a transponder configured to respond to electromagnetic radiation from an external source, and
a microchip operatively connected to the transponder and configured to activate in response to the electromagnetic radiation; and
an anti-migration device configured to receive at least a portion of the marker body and to engage tissue in which the marker body is implanted to resist movement of the marker body after implantation, wherein the anti-migration device includes a portion configured to transition between a tensioned state and an expanded state.

28. A marking system for implantation into tissue, comprising:
a marker body having an outer surface, the marker body including:
a transponder configured to respond to electromagnetic radiation from an external source, and
a microchip operatively connected to the transponder and configured to activate in response to the electromagnetic radiation; and
an anti-migration device configured to receive at least a portion of the marker body and to engage tissue in which the marker body is implanted to resist movement of the marker body after implantation, wherein the anti-migration device is formed from one or more wires, the one or more wires forming a spiral body comprising a radially expanded and protruding main portion and two radially fixed end portions, the radially fixed end portions of the spiral body being configured to grip the marker body.

29.-67. (canceled)
